SEQUENCE LISTING (19 sequences)

SEQ ID NO: 1 - DNA, 3170 bp, Arabidopsis thaliana
tctcaggtcggagctaaattttgagagtaaaataaaaaagcaaaagagagaaattgaaaaaaaaagaaagaaaaataatcctctcttgtttcggctagggtttcgtttttgtttcatcgataaactcaaaggagatggattcaacaaatggtaacggagctgatcttgaatcagcaaatggggcaaacgggagtggggttactgaggcattaccacctcctccaccagttatacctccaaatgtggaaccagttcgtgttaaaactgaacttgctgagaagaaggggccagttcgagttcctatggctcgaaaaggatttggaacaaggggccaaaagatccccttgttaacaaatcatttcaaagtcgatgtggctaatcttcagggtcatttcttccactacagtgtggctctattctatgatgatggtcgtcctgttgaacaaaagggtgttggaagaaaaatccttgacaaggtgcatcagacttaccattctgatctggatggtaaagagtttgcttatgacggtgagaagacgttgtttacatatggagctttgcctagtaacaagatggatttttctgtggtgcttgaggaagtatctgctacaagggctaatggaaacggaagccccaatgggaatgaaagtccaagtgatggtgataggaaaagactgcgtaggcctaaccggtccaaaaactttagagtggagatcagctatgcggccaaaattcctcttcaagctcttgctaatgcaatgcggggacaagaatcagagaattcccaggaggcaatacgggttcttgatatcatattgaggcaacatgctgctagacaaggttgcttgcttgttcgacagtcttttttccacaatgatccaaccaactgtgaaccagttggtggtaacatcttaggatgtaggggatttcactccagtttcagaacaacgcagggtggcatgtcacttaatatggatgttacaaccaccatgatcatcaagcctggtccagtggttgatttcctaattgctaaccaaaatgctagggacccttattcgattgactggtctaaggctaaacgaacccttaagaacctaagggtaaaggtcagcccctcaggccaagaattcaagataaccggattgagtgacaagccttgcagggaacaaacgtttgaattgaagaaaaggaacccaaatgaaaatggagagttcgaaactactgaagttacagttgctgactacttccgcgatacaaggcatattgatttgcaatattctgcggatttgccttgcatcaatgttgggaagccaaagcgacccacttacattcctctcgagctctgcgcgttggttccacttcagaggtacacaaaagcacttaccacgttccaaagatctgcccttgttgagaaatccagacagaaaccccaagagaggatgactgttctgtccaaagctctgaaagttagcaactatgatgcggaaccactcctgcgatcctgtggcatttcgatcagctccaactttactcaggtggagggtcgtgttctaccagctcccaagctgaaaatgggatgtggatctgaaacctttcccagaaatggtcgctggaacttcaacaacaaggaatttgttgagcccaccaaaattcaacgatgggttgttgtcaatttctctgctcgctgtaatgtacgtcaagttgttgatgatctgataaaaattggaggatcaaaaggaattgaaattgcttctccctttcaagtgtttgaggagggtaatcaattccgccgtgctcctcctatgattcgtgttgagaacatgtttaaggacatccaatcgaaactccctggtgtcccacaattcatactatgtgtgctccctgacaaaaagaacagtgatctctatggtccatggaagaaaaaaaacttaactgaatttggcattgttactcaatgcatggctccaacgcggcaacctaatgatcagtatcttactaacttacttctgaagattaatgcaaagcttggaggcctgaactcaatgttaagtgtagagcgtacacctgcgttcactgtgatttctaaggttccaaccattatccttgggatggatgtttcacatggatctcctggacagtctgatgtcccgtccatcgctgctgtggtgagttctagggagtggccactgatatccaaatatagagcatctgttcggacacagccttctaaggctgagatgattgagtcccttgtcaagaaaaatggaactgaagacgatggcattatcaaggagttgctggtagatttctacaccagctcgaataagagaaaaccagagcatatcataattttcagggatggtgtgagtgaatctcaattcaatcaggttctgaatattgaacttgatcagatcatcgaggcttgcaagctcttagacgcaaattggaacccaaagttccttttgttggtggctcaaaagaatcatcataccaagttcttccagccaacgtctcctgaaaatgttcctccagggacaatcattgacaacaaaatatgtcacccaaagaacaatgatttctacctctgtgctcacgctggaatgattggaactacccgcccaactcactaccacgtcctgtatgatgagattggtttttcagctgacgaacttcaggaacttgtccactcgctctcctatgtgtaccaaagaagcaccagtgccatttctgttgttgcgccgatctgctatgctcacttggcagctgctcagcttgggacgttcatgaagtttgaagatcagtctgagacatcatcaagccatggtggtatcacagctccaggaccaatctctgttgcacagctcccaagactcaaagacaacgtcgccaactccatgttcttctgttaaatgagcagccctacttggctcagtaagtaagtagtagttcaataggctatctgactttcagcttaggatctctctcggccttgaagccaggcatcgacactaaggcttggaatgttttttgtgactattatcgccagccttgtttgcctgttggggtttgaatatgcttttgtcccccttaacaattcggttgtttaattaaagacttattgtagtgaatatctaagttcaggaagttacccttttgtctaaatcgttctt

SEQ ID NO: 2 - protein, 924 aa, Arabidopsis thaliana
MDSTNGNGADLESANGANGSGVTEALPPPPPVIPPNVEPVRVKTELAEKKGPVRVPMARKGFGTRGQKIPLLTNHFKVDVANLQGHFFHYSVALFYDDGRPVEQKGVGRKILDKVHQTYHSDLDGKEFAYDGEKTLFTYGALPSNKMDFSVVLEEVSATRANGNGSPNGNESPSDGDRKRLRRPNRSKNFRVEISYAAKIPLQALANAMRGQESENSQEAIRVLDIILRQHAARQGCLLVRQSFFHNDPTNCEPVGGNILGCRGFHSSFRTTQGGMSLNMDVTTTMIIKPGPVVDFLIANQNARDPYSIDWSKAKRTLKNLRVKVSPSGQEFKITGLSDKPCREQTFELKKRNPNENGEFETTEVTVADYFRDTRHIDLQYSADLPCINVGKPKRPTYIPLELCALVPLQRYTKALTTFQRSALVEKSRQKPQERMTVLSKALKVSNYDAEPLLRSCGISISSNFTQVEGRVLPAPKLKMGCGSETFPRNGRWNFNNKEFVEPTKIQRWVVVNFSARCNVRQVVDDLIKIGGSKGIEIASPFQVFEEGNQFRRAPPMIRVENMFKDIQSKLPGVPQFILCVLPDKKNSDLYGPWKKKNLTEFGIVTQCMAPTRQPNDQYLTNLLLKINAKLGGLNSMLSVERTPAFTVISKVPTIILGMDVSHGSPGQSDVPSIAAVVSSREWPLISKYRASVRTQPSKAEMIESLVKKNGTEDDGIIKELLVDFYTSSNKRKPEHIIIFRDGVSESQFNQVLNIELDQIIEACKLLDANWNPKFLLLVAQKNHHTKFFQPTSPENVPPGTIIDNKICHPKNNDFYLCAHAGMIGTTRPTHYHVLYDEIGFSADELQELVHSLSYVYQRSTSAISVVAPICYAHLAAAQLGTFMKFEDQSETSSSHGGITAPGPISVAQLPRLKDNVANSMFFC

SEQ ID NO: 3 - DNA, 3018 bp, Arabidopsis thaliana
caccaaaacaaacaaaccctttcttcttcctctcataatccctctctctcttcttctctctcgctcgctgctctgtttagggtttttctcgttcgtctcttctataagtactagagatggattctgatgaaccgaatgggagtggattaccacctccaccacctttcgttccagcaaatcttgtccctgaagtggagcctgtaaaaaagaacattcttctcccaatggctcggcctcgaggcagcggctccaaaggacagaagattcctcttcttactaatcactttggagtcaagttcaacaaaccaagcggttacttctttcattacagtgttgctatcaattatgaagatggccgtccagtggaggctaaaggtatcggccgaaagattcttgacaaagttcaggagacctatcaaagtgatttgggtgccaaatactttgcttatgatggcgagaagactctcttcactgttggtgctcttcccagcaacaaacttgacttctctgttgttcttgaagaaataccttctagcagaaatcacgctggaaatgatacaaatgatgctgatagaaaaagatcaaggcgtcccaaccaaactaagaaattcatggttgagataagttatgctgcaaagatccccatgcaggctattgcaagcgctcttcaagggaaggagacagagaatcttcaagacgctctgagagtgctggatattattttgcgccagagtgcagctaggcaaggttgcctccttgttcgccagtcctttttccacaatgacgtaaagaactttgtacctattggtggaggtgtcagtggttgcagagggttccattcaagtttcagaactactcagggaggcttatccctgaatattgacacttcaactacgatgatagtacaacctggacctgtagttgatttcctgcttgctaaccagaacaagaaagatccatacggaatggactggaacaaggctcgacgtgtcctcaagaatctgagagttcaaattactctttccaatagagaatacaagataagtggactaagtgaacacagctgcaaagatcaactatttacatggaggaaacctaacgacaagggagaatttgaggaggttgagatcacagtgctcaattactataaagagcgtaacattgaagtgcgttattcaggtgacttcccttgcatcaatgttggtaagccgaagcgtcccacttacttccccattgagttctgtaatcttgtgtctctacagcgatacacaaaatcgcttaccaattttcagagggctgccctagttgaaaagtctaggcagaagccacctgaaaggatggcctcgctaaccaaaggtctgaaggacagcaattacaatgccgacccggtattgcaagatagtggtgttagcattatcaccaattttacccaagttgaaggccgtatcttaccaacaccaatgctgaaagtgggcaagggagaaaacctttccccaatcaaaggaaaatggaactttatgcgtaagacactcgctgagccaacgacggttactagatgggctgttgtgaacttctctgctcgctgtgatacaaatacacttattcgtgacttgattaaatgtggacgggagaaaggaattaatgtagagcctccattcaaggatgtcatcaacgagaatcctcagtttaggaatgcaccagctactgtgagagtagagaatatgtttgagcagataaaatccaaactcccaaagccgcctctgttccttctttgcatactcgctgaaaggaaaaactctgatgtttatggcccttggaaaaaaaagaatcttgttgatcttggaattgtgactcagtgcattgctcccaccagactgaacgatcagtatctcaccaatgttctcctgaagataaatgccaagcttggtggattgaattcgttgttagctatggagcgctcaccagcaatgccaaaagtaacgcaagttcctaccatcattgttgggatggatgtatcccatggttcccctggccagtctgatataccatcaattgctgctgttgtgagctcaagacaatggccactcatctcaaaatataaggcatgtgtacgcacacaatcacgcaaaatggaaatgattgataatctcttcaaacccgtcaatggcaaagacgaaggaatgttcagggaactcttgttagacttttactacagttcagagaataggaaaccagagcacatcattattttcagggatggtgtaagcgagtctcagttcaatcaagttcttaatattgaattggatcagatgatgcaggcatgcaagtttcttgatgatacgtggcatccgaagtttacagtgatagttgcccagaagaaccaccacacaaagttcttccagtctcgaggccctgataatgttcctccaggaacaatcattgacagccagatctgtcacccacgcaactttgatttctatctctgcgcccatgctggcatgattggaactacaaggccaacacattaccatgttctgtatgacgagattgggtttgccacagacgacctccaagaacttgtgcattctctgtcctatgtctaccagaggagcaccactgcgatctcagtcgttgcacctgtatgttacgctcatttggcagctgcacagatgggaactgtgatgaagtatgaagagttgtctgagacttcttcgagccatggaggaatcaccacacctggagcagtccctgtgccacctatgccgcagctgcacaataatgtttcaacctccatgttcttctgttgatggaacatggtccagtccacacgatagctgagtttatatgcttttgatggagaaccaacgaacttttgtttactcttctctgggtttggttcctcttttgtttttcgtcgaccctttaacattgaggagatactctttatattctgtcggtttttgtgggcgctgttggttgatactttattatgcttaatcagatttggtattttaaggc

SEQ ID NO: 4 - protein, 896 aa, Arabidopsis thaliana
MDSDEPNGSGLPPPPPFVPANLVPEVEPVKKNILLPMARPRGSGSKGQKIPLLTNHFGVKFNKPSGYFFHYSVAINYEDGRPVEAKGIGRKILDKVQETYQSDLGAKYFAYDGEKTLFTVGALPSNKLDFSVVLEEIPSSRNHAGNDTNDADRKRSRRPNQTKKFMVEISYAAKIPMQAIASALQGKETENLQDALRVLDIILRQSAARQGCLLVRQSFFHNDVKNFVPIGGGVSGCRGFHSSFRTTQGGLSLNIDTSTTMIVQPGPVVDFLLANQNKKDPYGMDWNKARRVLKNLRVQITLSNREYKISGLSEHSCKDQLFTWRKPNDKGEFEEVEITVLNYYKERNIEVRYSGDFPCINVGKPKRPTYFPIEFCNLVSLQRYTKSLTNFQRAALVEKSRQKPPERMASLTKGLKDSNYNADPVLQDSGVSIITNFTQVEGRILPTPMLKVGKGENLSPIKGKWNFMRKTLAEPTTVTRWAVVNFSARCDTNTLIRDLIKCGREKGINVEPPFKDVINENPQFRNAPATVRVENMFEQIKSKLPKPPLFLLCILAERKNSDVYGPWKKKNLVDLGIVTQCIAPTRLNDQYLTNVLLKINAKLGGLNSLLAMERSPAMPKVTQVPTIIVGMDVSHGSPGQSDIPSIAAVVSSRQWPLISKYKACVRTQSRKMEMIDNLFKPVNGKDEGMFRELLLDFYYSSENRKPEHIIIFRDGVSESQFNQVLNIELDQMMQACKFLDDTWHPKFTVIVAQKNHHTKFFQSRGPDNVPPGTIIDSQICHPRNFDFYLCAHAGMIGTTRPTHYHVLYDEIGFATDDLQELVHSLSYVYQRSTTAISVVAPVCYAHLAAAQMGTVMKYEELSETSSSHGGITTPGAVPVPPMPQLHNNVSTSMFFC

SEQ ID NO: 5 - DNA, 4948 bp, Arabidopsis thaliana
ttggacttagagaagagtttttttttcgttttctctgctttctcactttttcccgtttcctttgggaatacaaacggcggcttctcaaaagcctccgatttcgatttttttttcctctctttttttttgttctttctttagagtctcttcttcaacttcttctccggcatcttccgtcggagctaaggatcagaagtttttgtctgtgcactgtttcaattggatcattacatagtgaggattctctgttcgacgaaacgagaaactcttcaacatggaagacgattgtgaggagcttcaggttcccgtgggaactctaacctcgataggttttagcatttcaaacaacaatgatcgagataaaatgtctgtgctagaggttgaagcaccgaatcaggtgactgattctcggctaggattaccgaatccggattctgtctgcagaacctgtggcagcaaagatcgaaaagtttgcgaagggcatttcggggttataaacttcgcgtactcgataataaacccgtatttcctcaaggaggtagctgcgttgttaaacaaaatctgtccggggtgtaaatacattcgaaaaaagcagtttcagattaccgaagaccagcctgagagatgtagatactgcactttgaataccggttatcctctaatgaagtttagggtaactactaaagaagtcttcaggcgatccggaatcgttgttgaagtaaacgaagagagtctgatgaaactcaagaaacggggagtgttaacattgcctcctgactattggagttttttgccgcaagactctaatatcgacgagagctgtttgaaaccaacaaggcgaataataacgcatgcacaggtttatgctctgttattagggattgatcagaggctcatcaagaaggatatccctatgttcaactctcttggtctgacatcttttcctgttacaccaaatggttaccgtgtaaccgaaatagttcatcagttcaacggagctcggctaatatttgatgaacggactcggatctacaagaaactagtcggctttgagggaaacacacttgaattgagttctcgtgtgatggaatgcatgcaatattccagactcttttcggaaactgtgtcttcgagtaaagattccgcaaatccgtaccagaagaagtctgatacaccaaaactttgcggtctaaggttcatgaaagacgtgctgctcggtaaaagaagtgatcatacattccggacagtagttgttggtgatccgtccttgaagctcaacgagattggcatacccgagagtatcgcaaaaaggcttcaagtatcagagcatctcaaccagtgcaataaagaaaggcttgtcacttcctttgtccccacactgttggacaataaagagatgcatgtgagaagaggagatagattggtggctattcaagtcaatgacctccaaaccggagacaaaatattccggagtttaatggatggagacactgtgctgatgaacagaccgccttcgattcatcagcattcgcttattgcaatgacagttagaatcctcccaacgacttctgttgtctccttgaacccgatctgctgtttgccgttccgtggtgattttgatggagattgtctccacggttacgttcctcagtccattcaagcaaaggtcgagcttgatgagcttgttgctttggataagcagcttatcaacagacagaatggtcggaacttgctttctttaggacaagatagcttgacggcggcttatctggtcaatgttgagaagaactgttatcttaatcgagctcagatgcagcagcttcaaatgtattgcccatttcagcttcctccgcctgcaattatcaaagcttctccttcgagcactgagcctcaatggacgggaatgcagctattcggaatgctgtttcctcccgggtttgactacacttatcctctgaataatgtagttgtgagtaacggcgagcttttgtctttctctgaaggatctgcttggctcagagatggagaagggaatttcatcgagagattgctcaaacacgacaaagggaaagttcttgatatcatctattccgctcaagaaatgctatctcagtggttgctgatgcgcggtttgagtgtgtccttggcggatttgtacctttcctctgatctgcagtctcgaaaaaacttgactgaagagatatcatacgggttgcgggaagctgagcaagtttgcaacaagcaacagctaatggttgaatcttggagagatttccttgcagttaatggggaggataaggaagaggattcagtatcggatttggcccgattctgctatgagagacaaaaatccgctacattaagcgaacttgcagtcagtgcttttaaggatgcttacagagatgttcaggcactagcatataggtatggtgatcaatccaactcttttttgatcatgtcgaaagccggcagcaaagggaatatcgggaagctggttcagcacagcatgtgtatcggtctgcagaattcagcagtttctttgtcctttgggtttccgcgtgaactgacttgcgctgcttggaatgacccgaacagtccacttcgtggcgcaaagggaaaagacagtaccactactgagtcatatgttccttatggagtcattgagaactcgtttctaacgggtctgaatccattggaatcttttgttcattcagttacaagtcgtgacagttcattcagtggtaatgctgatctcccggggacactgagcagaagattaatgttcttcatgcgggacatatacgctgcttatgatggcactgtgagaaattctttcggaaaccagttagtccagtttacttatgagactgatggtcccgttgaagatatcacaggtgaggcacttggctcactttctgcttgtgcattatctgaggcagcttacagcgcgctggaccagccaatcagccttcttgaaacttcgccgcttctgaatcttaagaatgtattggagtgtggatcaaagaaaggtcaacgagaacagacaatgtctttgtatttgtctgaatacctttcaaagaaaaagcacgggttcgaatacgggtcacttgagatcaagaaccacttggagaaacttagtttctcagagatcgtttcaacatctatgataatattctctccaagttctaacacgaaagtaccgttaagcccgtgggtttgccattttcatatctctgagaaagtactgaagcggaaacaactgagcgcagaatccgttgtttcatctttaaacgagcagtacaagagcaggaacagagaactgaagcttgatatagttgacttagatatacaaaacacaaaccactgctcttcagacgatcaggcaatgaaggatgataacgtttgcatcacggtgactgtagttgaagcctcaaagcactcggttttggaactggatgctattcgtctcgttttgatcccttttcttcttgactctcctgtcaaaggtgatcaagggatcaaaaaggtgaatattttgtggactgaccgaccaaaagcgccaaaaaggaatgggaatcatttggcgggtgagctgtacttgaaggttacaatgtatggagatcggggcaaaagaaactgttggaccgctcttttagaaacctgccttcccatcatggatatgattgactggggccgaagccatcctgataatatccgacaatgctgctcagtttatggaatagatgctggacgcagcattttcgtagcaaatttggagtccgcggtgtcagatactggtaaggagatactaagagagcatctgcttctcgtagctgatagtctctctgttacgggagagtttgtggcattaaacgccaaaggttggagtaaacaaagacaggtcgagtccacgcctgcaccttttactcaagcatgcttctcaagcccaagtcaatgctttctcaaagctgctaaggaaggtgtcagagacgatcttcaagggtctatcgacgctttagcttggggaaaagttcctggtttcggaactggagatcagttcgaaatcatcatttcgccaaaggttcacgggtttactacaccggtagatgtgtatgaccttttgagtagtacaaagacgatgcgtagaacaaactctgcgcctaaatcagacaaagccacggtccagccttttggcttgctacattccgcattcctgaaagatatcaaggtactcgatgggaaagggattcctatgtcactgcttagaacgatctttacatggaaaaacatcgagttgctttcccagtcattgaaacgcattctccacagttacgagatcaatgaactgttgaatgagagggacgaaggacttgtgaagatggttctgcagttgcaccccaacagtgttgagaaaattggaccaggcgtcaaaggaatccgggtcgctaaatcgaagcacggagattcttgttgcttcgaggttgtaagaatagacgggacattcgaagacttctcataccacaagtgtgtcttgggagcaacaaagataattgccccaaagaaaatgaacttctacaagtcaaagtatcttaaaaatggtacactcgagtccggtggtttctccgaaaacccgtgaacagccaagtaagagtaaaaaaaaagactggctcttctgcggtataatatcgaacaatgtatgaagttgtgtgtaattttgtttaaaaagtgattagatagatataggggaaaagccttttggctttgctctaagcttctttttgctcctttcactatgtaataccttttgccttccacctaaaggcttaaaggttaattacttagtggaagcagcaacgagaagctggcaaaggaggcttctgttctgtttctgtttctgtatgtaaaccatgcaaattccggtttgactatgaaatatccggttatgttt

SEQ ID NO: 6 - protein, 1453 aa, Arabidopsis thaliana
MEDDCEELQVPVGTLTSIGFSISNNNDRDKMSVLEVEAPNQVTDSRLGLPNPDSVCRTCGSKDRKVCEGHFGVINFAYSIINPYFLKEVAALLNKICPGCKYIRKKQFQITEDQPERCRYCTLNTGYPLMKFRVTTKEVFRRSGIVVEVNEESLMKLKKRGVLTLPPDYWSFLPQDSNIDESCLKPTRRIITHAQVYALLLGIDQRLIKKDIPMFNSLGLTSFPVTPNGYRVTEIVHQFNGARLIFDERTRIYKKLVGFEGNTLELSSRVMECMQYSRLFSETVSSSKDSANPYQKKSDTPKLCGLRFMKDVLLGKRSDHTFRTVVVGDPSLKLNEIGIPESIAKRLQVSEHLNQCNKERLVTSFVPTLLDNKEMHVRRGDRLVAIQVNDLQTGDKIFRSLMDGDTVLMNRPPSIHQHSLIAMTVRILPTTSVVSLNPICCLPFRGDFDGDCLHGYVPQSIQAKVELDELVALDKQLINRQNGRNLLSLGQDSLTAAYLVNVEKNCYLNRAQMQQLQMYCPFQLPPPAIIKASPSSTEPQWTGMQLFGMLFPPGFDYTYPLNNVVVSNGELLSFSEGSAWLRDGEGNFIERLLKHDKGKVLDIIYSAQEMLSQWLLMRGLSVSLADLYLSSDLQSRKNLTEEISYGLREAEQVCNKQQLMVESWRDFLAVNGEDKEEDSVSDLARFCYERQKSATLSELAVSAFKDAYRDVQALAYRYGDQSNSFLIMSKAGSKGNIGKLVQHSMCIGLQNSAVSLSFGFPRELTCAAWNDPNSPLRGAKGKDSTTTESYVPYGVIENSFLTGLNPLESFVHSVTSRDSSFSGNADLPGTLSRRLMFFMRDIYAAYDGTVRNSFGNQLVQFTYETDGPVEDITGEALGSLSACALSEAAYSALDQPISLLETSPLLNLKNVLECGSKKGQREQTMSLYLSEYLSKKKHGFEYGSLEIKNHLEKLSFSEIVSTSMIIFSPSSNTKVPLSPWVCHFHISEKVLKRKQLSAESVVSSLNEQYKSRNRELKLDIVDLDIQNTNHCSSDDQAMKDDNVCITVTVVEASKHSVLELDAIRLVLIPFLLDSPVKGDQGIKKVNILWTDRPKAPKRNGNHLAGELYLKVTMYGDRGKRNCWTALLETCLPIMDMIDWGRSHPDNIRQCCSVYGIDAGRSIFVANLESAVSDTGKEILREHLLLVADSLSVTGEFVALNAKGWSKQRQVESTPAPFTQACFSSPSQCFLKAAKEGVRDDLQGSIDALAWGKVPGFGTGDQFEIIISPKVHGFTTPVDVYDLLSSTKTMRRTNSAPKSDKATVQPFGLLHSAFLKDIKVLDGKGIPMSLLRTIFTWKNIELLSQSLKRILHSYEINELLNERDEGLVKMVLQLHPNSVEKIGPGVKGIRVAKSKHGDSCCFEVVRIDGTFEDFSYHKCVLGATKIIAPKKMNFYKSKYLKNGTLESGGFSENP

SEQ ID NO: 7 - DNA, 3955 bp, Arabidopsis thaliana
aatttcttcacttctctttgactgcttcgcttaaccactgaaaaagtgtgccaagggttttctacgtcgaatctctccgcattctcagcgattttccggcgacgtttactctgcactcctccgacaccgccgttttactccatcgtgccagctttaagcaatcaagggtttttgttcgtacagtgtgttttgaggtatgccagatatggacattgatgtgaaggatcttgaagagttcgaggctactactggggagatcaatctatctgagctaggagaaggttttctgcagagtttctgcaaaaaagctgcaacttctttctttgataagtatggacttataagtcatcagctcaactcctacaactacttcattgaacacgggcttcagaatgtgtttcaatcctttggtgagatgcttgtggaaccgtcttttgatgttgtaaagaagaaggataatgattggagatacgcaacggtgaagttcggagaagtcactgtggagaagcctactttcttttcggatgacaaggagcttgagtttctcccatggcatgctaggcttcagaacatgacatactctgcaaggatcaaagtcaatgtccaagttgaggtgttcaagaatactgttgttaaaagcgacaaattcaagacaggacaagacaactatgtcgagaagaagatactggatgtcaaaaagcaggacattctaattggtagcattcctgtcatggtgaaatctatcctttgcaaaacaagcgagaaagggaaagaaaactgcaaaaagggggattgtgcctttgatcagggtggatatttcgtgataaagggggctgagaaggtgtttatagctcaagaacagatgtgcacaaagagactgtggatttctaattcaccatggacagtctccttcaggtccgaaaataaaagaaatagattcattgtgcgcctctcggagaatgagaaagcagaagactataagagaagggagaaagtactgacagtgtacttcttgtcgactgagattccagtctggctcctcttctttgcgctaggtgtttcgtcagacaaagaagccatggatctaattgcttttgatggtgatgatgcaagcattaccaacagtctcatagcttctatccatgtagctgatgcagtttgtgaagcttttcgctgtgggaacaatgctttaacatatgttgaacagcagatcaaaagcaccaaattccctcctgctgaaagtgtggatgagtgcctccatctgtatttgtttccaggcctccaaagtttgaagaagaaagctcgattcctgggctatatggtgaagtgccttctgaactcgtatgcgggaaaaagaaaatgcgaaaacagggacagtttccggaataagcgaattgagctcgctggagaactattggagagggagataagggtgcatctggcacatgctagaagaaagatgaccagggccatgcagaaacacctctcaggcgatggtgatttgaagcctattgagcattatttggatgcttctgttatcacaaatgggcttagtagagccttctctactggagcatggtctcatcctttcaggaagatggaaagggtttcaggtgttgtggctaatttgggtcgtgcaaatccattgcagactctgattgatctgaggagaacgcgacagcaagtcttatataccggcaaggttggagatgctagatatccgcacccctctcactggggcagagtatgctttttgtcaactccagacggtgaaaattgtggtcttgtgaagaacatgtctcttctgggacttgtgagcacccaaagtttggagtctgtggtggaaaagctcttcgcttgtggaatggaagagctgatggatgatacatgcacaccattgtttggcaaacataaagttcttctcaatggagactgggttggattatgtgcagattctgaatcctttgtcgcggagttaaaaagcaggcggcgccaaagtgaattacctcgtgagatggaaatcaagcgagataaagatgacaatgaggtaagaattttcactgatgctggtagactactccgacctctcttggttgtggaaaatctccaaaagttgaagcaagaaaaaccttcacagtatccttttgaccatcttcttgaccatgggattctcgagctgatcgggattgaggaagaagaagactgtaatacagcatggggaatcaaacagcttctgaaggaaccaaagatatacacacattgcgaattggacctgtcattcttgttgggtgtgagctgtgcagttgtcccatttgcaaatcacgaccatgggagaagagttctctaccagtcccagaagcactgccaacaagccattggattctcatcaacgaaccctaacatccgctgcgatacgctgtcccagcagctgttctatcctcagaagccactgttcaagacattggcgtcggagtgtcttaaaaaagaagtgctgttcaatggccagaacgcaattgttgctgtgaatgttcatctcgggtacaaccaagaggattccattgtgatgaacaaggcttcattggaacgtggtatgttccgttcagagcagattagaagctacaaagcagaggttgatgctaaagactcagagaagaggaagaagatggatgagcttgttcagtttggaaagacacacagcaaaatcggcaaagtagacagccttgaagatgacgggtttcctttcattggtgctaacatgagtactggcgatattgtcattggcagatgcaccgagtctggggctgatcacagtataaagctcaagcacactgagagaggaattgtgcaaaaagtggtattatcatctaatgatgaagggaagaattttgctgcggtttctctgagacaggttcgttctccatgccttggagataagttttccagtatgcatggccagaagggtgttttaggctacctagaggaacagcagaattttcctttcacgatccaaggcatagttcctgatattgtgataaacccgcacgctttcccttctaggcaaacaccaggtcaactcttggaggctgctctctccaaaggaatcgcttgtcctatacaaaaggagggtagctctgctgcatacaccaaattgacacgtcatgccactcctttctccactccgggtgtcactgaaatcaccgagcagcttcacagggccggcttttcaagatggggaaacgaaagggtctacaacggtagatcaggtgagatgatgcgttctatgatattcatgggcccaactttctaccagcgacttgtccacatgtcagaggacaaagtcaagttcaggaacactggaccagtccacccgctcacacgccagccagttgcagacaggaagagatttggcgggataaaatttggagaaatggagcgagactgcctaatagctcacggtgcatcagctaatctgcatgagcgtctcttcactctaagtgactcttctcagatgcacatctgcagaaaatgtaagacctatgcgaatgtgatcgagaggactccaagcagtggaagaaagattagagggccatattgtagagtctgcgtatcctcagaccatgtggttagggtctatgttccgtatggagctaagcttctgtgtcaggagctgttcagcatgggcatcactctcaacttcgacaccaagctatgctgattccccctctttattatgtaaatggcttattgccttaagaccatgttatgtgtagtttgcttcagtcccggttctggttagtagtataggttttggtttggttgattcggtaagggttatccgaaccgaagaaatcgtaaaaccgagccactgatgactgaactaacccgtaagtgttgcttttgtgagatttgactctttaaccgttaataattctcggatctaaagtaaagttttagg

SEQ ID NO: 8 - protein, 1172 aa, Arabidopsis thaliana
MPDMDIDVKDLEEFEATTGEINLSELGEGFLQSFCKKAATSFFDKYGLISHQLNSYNYFIEHGLQNVFQSFGEMLVEPSFDVVKKKDNDWRYATVKFGEVTVEKPTFFSDDKELEFLPWHARLQNMTYSARIKVNVQVEVFKNTVVKSDKFKTGQDNYVEKKILDVKKQDILIGSIPVMVKSILCKTSEKGKENCKKGDCAFDQGGYFVIKGAEKVFIAQEQMCTKRLWISNSPWTVSFRSENKRNRFIVRLSENEKAEDYKRREKVLTVYFLSTEIPVWLLFFALGVSSDKEAMDLIAFDGDDASITNSLIASIHVADAVCEAFRCGNNALTYVEQQIKSTKFPPAESVDECLHLYLFPGLQSLKKKARFLGYMVKCLLNSYAGKRKCENRDSFRNKRIELAGELLEREIRVHLAHARRKMTRAMQKHLSGDGDLKPIEHYLDASVITNGLSRAFSTGAWSHPFRKMERVSGVVANLGRANPLQTLIDLRRTRQQVLYTGKVGDARYPHPSHWGRVCFLSTPDGENCGLVKNMSLLGLVSTQSLESVVEKLFACGMEELMDDTCTPLFGKHKVLLNGDWVGLCADSESFVAELKSRRRQSELPREMEIKRDKDDNEVRIFTDAGRLLRPLLVVENLQKLKQEKPSQYPFDHLLDHGILELIGIEEEEDCNTAWGIKQLLKEPKIYTHCELDLSFLLGVSCAVVPFANHDHGRRVLYQSQKHCQQAIGFSSTNPNIRCDTLSQQLFYPQKPLFKTLASECLKKEVLFNGQNAIVAVNVHLGYNQEDSIVMNKASLERGMFRSEQIRSYKAEVDAKDSEKRKKMDELVQFGKTHSKIGKVDSLEDDGFPFIGANMSTGDIVIGRCTESGADHSIKLKHTERGIVQKVVLSSNDEGKNFAAVSLRQVRSPCLGDKFSSMHGQKGVLGYLEEQQNFPFTIQGIVPDIVINPHAFPSRQTPGQLLEAALSKGIACPIQKEGSSAAYTKLTRHATPFSTPGVTEITEQLHRAGFSRWGNERVYNGRSGEMMRSMIFMGPTFYQRLVHMSEDKVKFRNTGPVHPLTRQPVADRKRFGGIKFGEMERDCLIAHGASANLHERLFTLSDSSQMHICRKCKTYANVIERTPSSGRKIRGPYCRVCVSSDHVVRVYVPYGAKLLCQELFSMGITLNFDTKLC

SEQ ID NO: 9 - DNA, 3452 bp, Arabidopsis thaliana
atggtgtcagagacgacgacgaaccgatcaacggttaaaatctcaaacgttcctcaaaccatagtcgccgacgaactcctccggttcttagaactccacctcggcgaagatactgtattcgcactcgaaatcccaacaactcgtgacaattggaagccaagagacttcgctcgagtacaattcactaccctcgaagtcaaatctcgagcccagcttctctcttctcaaagcaagctccttttcaaaacccataacctaagactctctgaagcttatgatgatatcatccctcgtcctgtagatccaaggaaaaggctagacgacatcgttttgactgttgggtttcctgagtctgatgaaaaaaggttttgtgccctagaaaaatgggatggtgtgaggtgttggattttgactgagaagagaagagttgagttttgggtttgggaaagtggtgattgttataagattgaagttaggtttgaggatattattgaaactcttagttgttgtgttaatggtgacgcttctgagattgatgcatttcttctcaagctgaaatatggaccgaaggtatttaaaagagtaacagttcacatagccacaaagtttaagtctgatcggtacagattctgcaaggaggattttgatttcatgtggattcggacaactgatttctctggttcgaaatcaattggtacatcaacttgcttttgtttggaagtccacaatggctcaacgatgttagacattttctcgggcttaccttactaccgagaagacactttgagtctaacttatgtggatgggaagacctttgcttctgcagctcaaattgttcccctcttgaatgccgccattttggggttagagtttccatatgagatccttttccaactcaatgcgcttgttcatgcgcagaaaatcagtctttttgctgcgtcagatatggaactgatcaaaatccttcgtggtatgagtttggaaaccgcattggtgatcctcaagaaacttcaccagcaaagttctatatgttatgatcctgtcttctttgtcaagactcagatgcaatctgtggttaagaagatgaagcattcccctgcatcagcttataaaagattgaccgagcagaacatcatgagttgtcagagagcttacgttacaccctcaaagatctatctgttgggtccagagcttgagactgcaaattatgttgtgaagaactttgcagagcatgtctcggatttcatgagagttacttttgtggaagaagattggagcaagcttcccgcaaatgctctctctgtgaactccaaggaaggctattttgtgaagccctctagaaccaacatttataacagggtgttgtcaatccttggagaagggattaccgttgggcctaagaggtttgagttcttggctttttcagcgagccaactgcgaggaaattcggtctggatgtttgcttctaacgagaaagtaaaagcggaagatataagagaatggatgggttgtttccgtaaaatccgaagcatttccaaatgtgctgctaggatgggtcagttgttcagtgcttcgcggcaaacacttattgtccgtgcacaagatgtggagcagattcccgacatcgaagtgacaactgatggtgctgattactgcttctcggatggcattgggaaaatttcacttgcatttgccaagcaagttgcacagaagtgtggattgagtcatgtcccttctgcctttcaaattcgatacggtggctacaaaggtgtgattgctgttgaccgcagttccttccgaaagttgtctctgcgtgatagtatgcttaaatttgactcgaacaacaggatgctgaacgttaccaggtggacagagtcgatgccttgcttcttaaaccgggagatcatttgccttttgtcgacccttggaatagaagatgcaatgtttgaggcgatgcaagcggtgcatttatctatgcttgggaatatgcttgaagaccgcgatgcagcacttaatgttctgcagaaattgagcggagaaaattccaagaatttgctagttaagatgctgcttcaaggatatgcaccgagttcagaaccttacctctcaatgatgcttcgtgtgcaccacgagagccagctttctgaactaaagagcagatgcaggatacttgtaccgaaaggacggatcttgatcggttgcatggatgaaatgggtatcctggagtatggccaagtgtatgttcgtgtaaccctgactaaagcggaactgaaatctcgcgatcagagctactttcgcaagattgatgaggaaacatctgtggttattgggaaagtggtcgtgacgaaaaacccatgtcttcaccctggagatattagagttcttgatgctatatatgaggtccatttcgaagaaaagggatatctcgactgcatcatctttcctcagaagggagaaagaccacatccaaatgaatgttctggtggcgatctcgacggagaccagttttttgttagctgggatgagaagattatacctagcgaaatggaccctccaatggactatgctggaagcaggcctcgtctaatggatcatgacgtcacgttagaggaaatccacaaattttttgtggattatatgataagcgacacgcttggggtgatctcaactgcacatttggttcacgcagaccgtgaccctgaaaaagcccggagccagaagtgtctagagttagcaaatcttcactctagggctgttgattttgcaaaaactggagctccagctgagatgccttatgccttaaagcccagagagttccctgattttctggaacggtttgagaaaccaacatacatctctgagtctgtgtttgggaaactataccgtgctgtgaagagctctctagcacagagaaagccagaagctgagagcgaggacacggtagcttatgatgtgacacttgaagaagctggctttgagagctttatagagacagcgaaagcccatagagacatgtatggtgagaaactgacctcgttgatgatatactatggagctgctaacgaagaggagattctcacaggcattttgaaaaccaaggagatgtatctggcgagagataaccggaggtatggtgatatgaaggatagaattacgctttccgtgaaagatttgcataaagaggccatgggatggttcgagaaaagctgcgaggacgaacaacagaagaagaagctagcatcggcgtggtactatgtaacatacaatccaaaccatcgcgatgagaagttgacattcttgagtttcccatggatcgtaggtgacgttttgcttgatataaaggctgaaaatgcacagagacagagcgttgaagagaaaacaagtggacttgtatccatttgattgccctatataacacttggtgtagtagaattgctagttaaaggactata

SEQ ID NO: 10 - protein, 1133 aa, Arabidopsis thaliana
MVSETTTNRSTVKISNVPQTIVADELLRFLELHLGEDTVFALEIPTTRDNWKPRDFARVQFTTLEVKSRAQLLSSQSKLLFKTHNLRLSEAYDDIIPRPVDPRKRLDDIVLTVGFPESDEKRFCALEKWDGVRCWILTEKRRVEFWVWESGDCYKIEVRFEDIIETLSCCVNGDASEIDAFLLKLKYGPKVFKRVTVHIATKFKSDRYRFCKEDFDFMWIRTTDFSGSKSIGTSTCFCLEVHNGSTMLDIFSGLPYYREDTLSLTYVDGKTFASAAQIVPLLNAAILGLEFPYEILFQLNALVHAQKISLFAASDMELIKILRGMSLETALVILKKLHQQSSICYDPVFFVKTQMQSVVKKMKHSPASAYKRLTEQNIMSCQRAYVTPSKIYLLGPELETANYVVKNFAEHVSDFMRVTFVEEDWSKLPANALSVNSKEGYFVKPSRTNIYNRVLSILGEGITVGPKRFEFLAFSASQLRGNSVWMFASNEKVKAEDIREWMGCFRKIRSISKCAARMGQLFSASRQTLIVRAQDVEQIPDIEVTTDGADYCFSDGIGKISLAFAKQVAQKCGLSHVPSAFQIRYGGYKGVIAVDRSSFRKLSLRDSMLKFDSNNRMLNVTRWTESMPCFLNREIICLLSTLGIEDAMFEAMQAVHLSMLGNMLEDRDAALNVLQKLSGENSKNLLVKMLLQGYAPSSEPYLSMMLRVHHESQLSELKSRCRILVPKGRILIGCMDEMGILEYGQVYVRVTLTKAELKSRDQSYFRKIDEETSVVIGKVVVTKNPCLHPGDIRVLDAIYEVHFEEKGYLDCIIFPQKGERPHPNECSGGDLDGDQFFVSWDEKIIPSEMDPPMDYAGSRPRLMDHDVTLEEIHKFFVDYMISDTLGVISTAHLVHADRDPEKARSQKCLELANLHSRAVDFAKTGAPAEMPYALKPREFPDFLERFEKPTYISESVFGKLYRAVKSSLAQRKPEAESEDTVAYDVTLEEAGFESFIETAKAHRDMYGEKLTSLMIYYGAANEEEILTGILKTKEMYLARDNRRYGDMKDRITLSVKDLHKEAMGWFEKSCEDEQQKKKLASAWYYVTYNPNHRDEKLTFLSFPWIVGDVLLDIKAENAQRQSVEEKTSGLVSI

SEQ ID NO: 11 - DNA, 3591 bp, Arabidopsis thaliana
atggggtcagagggaaatatgaagaagtcggttgtgacacaagtaagcattggtggatttggggaatccactactgcaaaacaactcacagattaccttgaagatgaagtaggaattgtttggcgttgcagattaaaaacttcgtggactcctcctgggtcttatcctaattttgaaatcgcagacacttcaaatatccccagtattgatgaatataagaaagtggagcctcatgcctttgtccatttcgcagtttttgaatcagctggtcgtgccatggatgctgcagggcaatgcaacctcatcttggatggccagccgttaaaagtcagtttgggtcctaagaatccatattcccttaatcaaagaagaagaacgactgtaccttataagttggctggtattacacttgagattgggacattggtttctcgggatgatttttttgtttcttggagagctgaaggggttgatttcctggtggatccttttgacaacacgtgcaagttttgtttcagaaagagcactgccttctctttcaaggatgcagtgatgcatgctgtgatcaattgtgattataagttggagttattggtgagagatatacaaacagtcaggcagtataaaaccttgcatggctttgtgcttattttgcagctggcttcttcaccccgtgtctggtacagaactgctgatgatgatatttacgatactgtccctggcgatctcttggatgatgatgatccttggatccgtaccactgattttactcaagttggggcaattggtcgatgtcattcatatcgagtgctcatatctccgcggtatgaaaataaactgagaacagccttagattattttaggatgcggagggtgcaagaggaacgtgtgaggtggcctccccgtatccgtaatgagccttgttttggggagcctgtgtcagatcatttcttctgcattcatcacaaggaaggaatctcttttgagatcatgtttctagtaaattcggtattgcacaggggtgtctttaaccagtttcagttgactgagcgattctttgatcttctaagaaaccaacccaaggacgtcaatatagcttctctcaagcatctctgtacctataaacgaccagtttttgatgcgtacaagaggttgaagcttgttcaggaatggattcagaaaaatccaaagcttttagggagtcatgaacaatctgaggatatctctgagatcagaagactagtaattaccccaaccagagcctattgcctacccccagaagttgagctctccaacagggtactcaggagatacaaagctgttgctgaaagatttttgcgggtaactttcatggatgaaagtatgcagaccataaattcgaatgttctctcttactttgttgctcctattgtgaaggatttgacatcaagttctttctcccagaagacctacgtttttaaaagagtgaagagcatattaaccgatgggtttaaactatgtggtagaaaatacagttttctagcattctcagccaatcaactgagagaccgctctgcatggttctttgctgaagacgggaaaacacgtgtgtcagatataaaaacatggatggggaagttcaaagacaagaatgtggcaaaatgtgctgctaggatgggcctgtgcttctcctccacatatgccactgtagatgtcatgcctcacgaggttgacactgaggttccagatattgagagaaatgggtatgttttctctgacggaattggtacaatcacacctgacctcgctgacgaagtaatggagaaacttaagttggatgtgcactacagcccttgtgcttatcagatacgttacgcaggtttcaaaggggttgttgctcgttggccatcaaaaagtgatggaatcaggctagcccttcgagacagtatgaagaagttcttttccaaacatacgatcttggagatctgttcctggacgaggtttcaacctgggttcttaaatcggcagataattacccttctatccgtactaggtgttccggatgaaatattctgggatatgcaggaatccatgctctataaactgaaccgcatccttgatgatacagatgtggcatttgaagttctcacggcatcatgtgctgaacagggaaacactgcagctatcatgcttagtgcaggtttcaaaccaaaaaccgagccgcatctacgcgggatgttgtcttcagtcagaattgcacaactctggggtctcagagaaaaatctcgtatttttgttacttcaggaaggtggctaatgggttgcctagacgaagcagggatacttgaacatggccaatgctttattcaagtctctaaaccgtctatagaaaattgtttctccaaacatgggtctcgttttaaggagacaaagacagatctggaagtagttaaaggctatgtagccattgctaagaatccttgtcttcacccaggggatgtaaggattttagaagctgttgatgtaccccagctgcatcacatgtatgactgccttattttccctcagaaaggtgataggccgcatacaaacgaagcttctggcagtgaccttgacggggacctgtactttgtggcttgggatcagaaactcatccctcccaacaggaaaagctatccggccatgcattatgatgcagctgaagagaagagtttaggccgtgctgtcaaccaccaggacataatcgatttctttgcaagaaacttggcgaatgagcagttgggcacaatttgcaatgcacacgtcgttcatgctgatagaagtgagtatggagccatggacgaagaatgtttgctactggcagaactagctgccactgcagttgatttcccaaagacagggaaaattgtgtcaatgcccttccacctaaaaccaaaactctacccagatttcatgggaaaagaagactaccaaacttacaagtcgaacaaaatcttgggtcggctttacagacgggtaaaagaggtttatgatgaagatgcagaagcttcctcagaagaaagcacagacccaagtgccatcccttatgacgctgttcttgaaataccgggatttgaagatttgatccctgaggcatggggtcacaaatgtttgtacgacgggcaactcattggtcttcttgggcaatacaaggtgcagaaagaggaagagattgtgacgggtcacatctggtccatgcccaaatacacaagcaagaaacaaggcgaactgaaagaaagactgaagcattcttataattcccttaaaaaggagttcaggaaagtatttgaggaaacaatccctgaccatgaaaatctcagcgaggaggaaaaaaacatcttgtatgagaagaaggcttcagcttggtatcatgtaacttaccatcccgagtgggtgaagaagtctttagagctgcaagatccagatgagtccagtcatgcggcgatgctgagttttgcttggattgcagctgattatcttgcaagaatcaaaatccggtcacgggaaatgggaagtatcgactcagccaagcctgttgattctttggctaagtttcttgctcagcgtctctaa

SEQ ID NO: 12 - protein, 1196 aa, Arabidopsis thaliana
MGSEGNMKKSVVTQVSIGGFGESTTAKQLTDYLEDEVGIVWRCRLKTSWTPPGSYPNFEIADTSNIPSIDEYKKVEPHAFVHFAVFESAGRAMDAAGQCNLILDGQPLKVSLGPKNPYSLNQRRRTTVPYKLAGITLEIGTLVSRDDFFVSWRAEGVDFLVDPFDNTCKFCFRKSTAFSFKDAVMHAVINCDYKLELLVRDIQTVRQYKTLHGFVLILQLASSPRVWYRTADDDIYDTVPGDLLDDDDPWIRTTDFTQVGAIGRCHSYRVLISPRYENKLRTALDYFRMRRVQEERVRWPPRIRNEPCFGEPVSDHFFCIHHKEGISFEIMFLVNSVLHRGVFNQFQLTERFFDLLRNQPKDVNIASLKHLCTYKRPVFDAYKRLKLVQEWIQKNPKLLGSHEQSEDISEIRRLVITPTRAYCLPPEVELSNRVLRRYKAVAERFLRVTFMDESMQTINSNVLSYFVAPIVKDLTSSSFSQKTYVFKRVKSILTDGFKLCGRKYSFLAFSANQLRDRSAWFFAEDGKTRVSDIKTWMGKFKDKNVAKCAARMGLCFSSTYATVDVMPHEVDTEVPDIERNGYVFSDGIGTITPDLADEVMEKLKLDVHYSPCAYQIRYAGFKGVVARWPSKSDGIRLALRDSMKKFFSKHTILEICSWTRFQPGFLNRQIITLLSVLGVPDEIFWDMQESMLYKLNRILDDTDVAFEVLTASCAEQGNTAAIMLSAGFKPKTEPHLRGMLSSVRIAQLWGLREKSRIFVTSGRWLMGCLDEAGILEHGQCFIQVSKPSIENCFSKHGSRFKETKTDLEVVKGYVAIAKNPCLHPGDVRILEAVDVPQLHHMYDCLIFPQKGDRPHTNEASGSDLDGDLYFVAWDQKLIPPNRKSYPAMHYDAAEEKSLGRAVNHQDIIDFFARNLANEQLGTICNAHVVHADRSEYGAMDEECLLLAELAATAVDFPKTGKIVSMPFHLKPKLYPDFMGKEDYQTYKSNKILGRLYRRVKEVYDEDAEASSEESTDPSAIPYDAVLEIPGFEDLIPEAWGHKCLYDGQLIGLLGQYKVQKEEEIVTGHIWSMPKYTSKKQGELKERLKHSYNSLKKEFRKVFEETIPDHENLSEEEKNILYEKKASAWYHVTYHPEWVKKSLELQDPDESSHAAMLSFAWIAADYLARIKIRSREMGSIDSAKPVDSLAKFLAQRL

SEQ ID NO: 13 - DNA, 2145 bp, Arabidopsis thaliana
agtttattttcttcctccggagtcctgactcactactctcactctccggcgctttaaacttacgttctccgtcgtttactctgttgtaaaaatgagttctagggctggtccaatgtctaaggaaaagaacgttcagggtggttataggcctgaggttgaacagttggttcaaggtttggcagggacgagactggcttcttcacaagatgatggaggagagtgggaggtcatttccaagaagaacaagaacaaaccaggaaacacttctggaaaaacttgggtttctcagaattcgaatcctcctagagcttggggtggtcagcagcaagggagaggtagcaacgtatctgggagaggaaacaatgtatccgggagaggtaacggcaatggtcggggcattcaagctaacatatctggtcggggacgagcgttgagcagaaagtatgataacaactttgtggcacccccacctgtatctcgccctcctttggaaggaggatggaattggcaggcaagaggaggttctgctcagcacacagctgtgcaggagtttcctgacgtggaggatgatgtggataatgcttctgaggaagagaatgattccgatgctttggatgattctgatgacgaccttgcaagtgatgattatgactcggatgtgagtcaaaagagccatggatcacgaaagcagaataagtggttcaaaaagttctttggcagcttggatagcttgtcgatcgagcagataaatgaaccacagaggcagtggcattgtccagcttgtcagaacggacctggtgccatcgattggtataacctgcaccctctactagctcatgcgaggacaaaaggagctaggcgagttaagctccatagagaattggctgaagttttagaaaaggatctacagatgagaggcgcatctgtcattccttgtggtgagatttatgggcagtggaagggtttgggtgaggatgaaaaggattatgaaattgtctggcctccaatggtcatcatcatgaatactagactggataaggacgataacgataagtggctcggcatgggcaaccaagagctgctggaatacttcgacaagtatgaggctcttagagcacgccattcctatggtccacagggccatcgtgggatgagtgttctgatgtttgagagcagtgccactggctatttggaggccgaacgcctccaccgggagttagctgagatggggttagatagaattgcctggggtcagaagcgcagtatgttttctggaggtgttcgccaactgtatggcttccttgcaacgaagcaagatctggacatattcaatcaacactctcaaggcaaaacaaggctgaaattcgagttgaaatcataccaagagatggttgtaaaggagctgaggcagatctctgaggacaatcagcagctgaactactttaagaacaagctctcaaaacagaacaagcacgccaaggtgcttgaggaatctctggaaattatgagcgagaagctgcgtagaactgcagaggataatcggatcgtgagacagagaactaagatgcagcatgaacagaacagggaagagatggatgcacacgacaggtttttcatggattcaatcaaacagatccatgaaagaagagacgcaaaggaggagaatttcgagatgttgcagcagcaggaacgtgccaaggttgttggccagcagcagcagaacattaatccctctagcaatgacgattgccgaaagagagctgaggaagtgtcaagcttcatcgagtttcaagagaaagagatggaggagtttgtggaagagagggagatgctgataaaagatcaagagaagaagatggaagacatgaagaagaggcatcacgaggagatatttgatctggagaaagaatttgatgaggctttggaacagctcatgtacaagcatggccttcacaatgaagatgattgagacaaaagtctggtacacaagacaagactaagtttctttgttttgcttttggtatgtcggaaagtaggagatctgagagactccatttaaatactaggacaaatctaaggagattatagattattatcctccaatttttagtagacggatctaaggaagcattaagttcttgtgac

SEQ ID NO: 14 - protein, 625 aa, Arabidopsis thaliana
MSSRAGPMSKEKNVQGGYRPEVEQLVQGLAGTRLASSQDDGGEWEVISKKNKNKPGNTSGKTWVSQNSNPPRAWGGQQQGRGSNVSGRGNNVSGRGNGNGRGIQANISGRGRALSRKYDNNFVAPPPVSRPPLEGGWNWQARGGSAQHTAVQEFPDVEDDVDNASEEENDSDALDDSDDDLASDDYDSDVSQKSHGSRKQNKWFKKFFGSLDSLSIEQINEPQRQWHCPACQNGPGAIDWYNLHPLLAHARTKGARRVKLHRELAEVLEKDLQMRGASVIPCGEIYGQWKGLGEDEKDYEIVWPPMVIIMNTRLDKDDNDKWLGMGNQELLEYFDKYEALRARHSYGPQGHRGMSVLMFESSATGYLEAERLHRELAEMGLDRIAWGQKRSMFSGGVRQLYGFLATKQDLDIFNQHSQGKTRLKFELKSYQEMVVKELRQISEDNQQLNYFKNKLSKQNKHAKVLEESLEIMSEKLRRTAEDNRIVRQRTKMQHEQNREEMDAHDRFFMDSIKQIHERRDAKEENFEMLQQQERAKVVGQQQQNINPSSNDDCRKRAEEVSSFIEFQEKEMEEFVEEREMLIKDQEKKMEDMKKRHHEEIFDLEKEFDEALEQLMYKHGLHNEDD

SEQ ID NO: 15 - DNA, 2748 bp, Glycine max
atggattcatttgagccagatggaaatgggaaggagtcactgccaccaccacctcctgttgttccctctgatattgtacctctcaaagcagaggaggtgctctgtacccctaccgagcataataagaaaaaggcttcccgacttccaatagccagatctggtctgggatcaaaaggaaataaaatacaattactaaccaatcacttcaaagttaatgttgctaaaaatgatgggcatttcttccattatagtgtggcttttacttatgaagatggacgccctgtagaaggtaagggtgtagggagaaagataatagatagggtgcaggagacatatcattctgacttaaatggtaaggactttgcatatgatggggagaaaagtctgtttactgttggctctcttcctcaaaacaagcttgagtttgaagttgttcttgaggatgtcacctctaacaggaataatggcaattgcagccctgatggtctaggggacaatgagagtgacagaaagaggatgcgacgtccttatcgttcgaagtcattcaaagtagagataagctttgctgcaaaaattccaatgcaggccattgccagtgccttacgcgggcaagagactgagaattttcaagaagccatcagagttcttgatatcattttgaggcagcatgctgctaagcaaggctgcttacttgtacgccaatcctttttccacaataatccaaataattttgctgatgtaggaggtggtgtcctaggctgtagaggattccactcaagctttagaactacacagagtggcctgtctcttaacatagatgtgtcaactacaatgataatttctcctgggcctgtggtggatttcttaatttccaatcaaaatgtgagagatccttttcaacttgactgggctaaggccaaaaggaccctaaaaaatctgaggattaaaactagcccatccaatcaagaattcaaaatttctgggctcagtgaactcccatgcagagagcagacttttactttgaaaggtaaaggtgggggggatggtgaagatggtaatgaggaaatcactgtatatgattattttgttaaggttcgtaagatagatctccgatactctgctgaccttccatgtatcaatgttggcaagcctaaacgaccaacatttttccccattgaggtttgtgaattggtatcattgcaacgatatacaaaagctctgtccacgcttcaaagggcttcattagtggagaagtcgaggcagaagccacaagagaggatgaaaattttgtctgatgcactgagaacaagcaactatggtgctgaacctatgctccggaattgtggaatttctataagcactggcttcactgaagtggagggccgggtgttgcctgcaccaaggttgaagtttggcaatggtgaggatctcaatcctaggaatgggagatggaatgtcagcagagtgaaatttgtggaaccatcaaagatagaaagatgggctgttgctaacttttctgcacgctgtgatgtacgaggacttgtacgggacctcattagaattggagatatgaaaggaattactatagaacaaccatttgacgtgtttgatgagaatccacagtttaggcgtgccccccctatggttagagtggagaaaatgttcgagcatatccaatctaaacttcctggggctcctcagttccttctctgtttgcttcctgatcggaaaaattgtgatatttatggtccatggaaaaagaagaatcttgctgattttggaatcataaatcagtgtatgtgtcctttaagggtcaatgaccagtacctgactaatgttatgttgaagatcaatgccaagcttggtgggttgaattcattgttaggcgttgaacattctccttctcttcctgttgtttccaaagctcccaccctcattctgggaatggacgtgtcacatggctcacctgggcagactgacattccttcaattgctgcggtggtcagctctagacactggcctctgatatcaaagtatagggcatgtgttcgtacgcaatctgcaaagatggaaatgattgataatttgttcaagctagtatctgaaaaggaagatgaaggcatcataagggaacttttgcttgatttctatacaacttctgggaggagaaaaccggaaaatataatcatattcagggatggggttagtgagtcacaattcaatcaagttttgaatattgaactcgatcgaatcattgaggcttgcaaatttctcgatgaaaattgggagccaaaatttgtggtaattgttgctcagaagaaccaccacactagatttttccagcctggctctcccgacaatgtcccacctggaactgttatcgacaataaaatttgtcatcccagaaattatgatttctacctatgtgcacatgctggaatgataggaactagtaggcctacccattatcatgtgctgcttgatcaggttggtttctctccggatcagctgcaggagcttgtccattcattatcatatgtgtatcagaggagcactactgccatttctgttgttgctccaatatgctatgcgcacttggctgctactcagttggggcagttcatgaaatttgaggacaaatctgaaacatcttcaagccatggtggattgagcggtgcaggtgctgttcccgtccctcagttgcctcccttgcaggagaatgtccgcaacacaatgttcttttgttga

SEQ ID NO: 16 - DNA, 2751 bp, Vitis vinifera
atggattctggggaagatggaaatggggcacaagatgctttgccacctcccccacctgttccgccaaatgttgttccaataaaagctgattcaccagttaagaaaaaggtcgcacgtgttccaatagctcgccgtggctttgcatccaaggggcaaaaaatagcactaactactaaccacttcaaagttaatgttactggtgctgatggtcacttcttccattacagtgtttccctttcatatgaagatggccgtcctgttgatggtaagggaattggaagaaaggttatcgatagagttcatgagacatatgatagcgagttaggtggaaaggactttgcttatgatggggagaagagtttgttcacagttggtcctcttccacgaaacaaacttgagttcactgttgtgcttgaggatgtttcatcaaataggaataatggcaatggaagtcctgatcgtggtagtccgaatgagagtgatcgaaaaaggatgcggcgtccttaccagtcaaagacttttaaagtagagattagctttgctgctaaaataccaatgcaggcaattgccaatgcactacgtggtcaagaatcagaaaactctcaagaagcacttagagttttggatatcattttaaggcagcatgcatcaaaacagggttgcctccttgttcgtcaatccttttttcacaatgatccaaaaaatttcattgatttgggagggggcgttcttggctgcagaggattccattcaagttttcgaaccacccaaggaggcttatcactgaatattggcaagttattgattttattttatgtatctactaccatgatagtgcaacctgggccagtggttgattttttaattgccaatcaaaatgcgagggatcctttttccctggactgggctaaggccaagaaaatgctaaaaaatctgagggtgaagacaagcccctcaaacaccgagtacaaaataactggactgagtgagaagccttgcaaggagcagttgtttacgcttaagcaaagaaatgggaaggatgagaatggcgaggcccaaacgattgaagtgactgtttttgattattttgttaatcatcgccgcatagaactacgttattctgcagatttaccttgcattaatgttgggaagccaaaacgaccgacttacttccctatagagctttgtaccttggtgtcgttacaacgttatactaaagcgttgtccactcttcaaagagcttcactggtggaaagatcaaggcaaaaaccacaagaaaggataggagttttgactaatgctttgagaagcaacaattatgatgctgagcctatgctacgttcctgtggcatttcaataagcagagacttgacccaaattgaaggccgtgttctggcagctccaaggttgaaagttggtaatggggaggatttctttccacgaaatgggcggtggaattttaacaataagaaactggtggagcccacaaagatagaacgttgggctgtggtcaacttctcggctcgctgtgatattcgaaacctcgtccgagaactgatcaaatgtggaggaatgaaaggaattcacattgatcctccatttgatgtatttgaagagaatccacaatctcgacgagccccacccattgttagggtggagaaaatgtttgaggagatacagtctaaactccctggagctcctcagttccttctctgtctacttccagagaggaaaaactctgatctatatggtccttggaaacgaaagaatctttctgaatatggaattgtgactcaatgcattgctcctacaagggttaatgatcaatatcttacgaatgttctcctaaagattaatgcaaaacttggtggattaaattctatgctagcagtagaacattccccttctattccaattgtttcgaagggacccaccataatccttgggatggatgtgtctcatggttctcctggacaatctgatgtaccatctattgctgcggttgtcagctccaggcagtggccactgatttcgcgctatagagcatcagttcgtacacaatctccaaaggttgagatgattgattctctgtataagcgagtatctgaaactgaagatgaaggcataattagagagcttttgctagacttttatgtgagttcaggcaaaagaaaacccgatcagattatcatattcagggatggagtcagcgagtctcagttcaatcaagttctgaacatcgaactggatcaaattattgaggcctgcaagttccttgatgagaagtggtctcccaaatttgtggtgattgttgcgcagaaaaaccatcataccaagtttttccaacatggatctcctgataacgtcccacctggcacagtcatagacaacaaagtttgtcatccacggaacaatgacttttatctctgtgcacatgctggaatgattggtactaccaggccgacgcattaccacgttctattggatgaagttggtttctcttcggatgatcttcaggagcttgtgcattctttatcctatgtgtaccaaaggagcaccactgccatttccgtagttgctcccatatgctatgcccacttagcagctactcagatgtctcagttcatgaagtttgaagacacgtcagagacatcctcaagccaaggtggactgacgtcagcagggcctgttccagtgcctcaactccccaaattgcaggagagtgtctgcaattcgatgttcttttgctga

SEQ ID NO: 17 - DNA, 3140 bp, Populus trichocarpa
gatatggagtcagctgatgaacaaaatggaaatgggtcacaggaagccctcccacctccccctcctgatgttccaccaaatgttgttccagttaaagctgaacctgagccagtcaagaaaaaacctctgcgggttccaatagccaggcgtggccttggatccaaaggccaaaagatgcctctattgaccaatcactttaaagtcaatgttactaatactgagggttacttctttcactactgtgtttcccttgcttatgaagatggccgccctgttgatggtaagggtgttggaagaaaggtgattgatagggtgcatgaaacttatgataccgagtttggaaaggattttgcttatgacggtgaaaagagcttgttcactgttggtccccttcctcgcaacaagcttgagttcacggttgtgcttgaggatgtagtatctaacagaaataatggaaatgcaagccctgatggtcatggaagtccaaatgagggtgaccgaaagaggttgcgccgtccctatcactccaagacattcaaagtggagatcagttttgctgcaaaaatccccatgcaagccattgcaaatgccttgcgtggtcaggaatcagagaattcccaagaagcctttagagtgctggatattatattgcgacagcatgccgccaagcagggctgcctccttgtgcgccaatccttcttccataatgatccaaaaaattttgtggatttgggaggcggtgttcttggctgcagaggtttccactcaagttttagaacatctcagggaggcttgtctcttaatattgatgtgtctacgaccatgataatacagccaggtcctgtggtagattttctaattgccaaccaaaatgtgagagatccattttcacttgactgggcgaaggcgaaacgaatgctcaaaaatctgagggttaaggcaagtccttccaatcaagagtacaagataactgggttgagtgagaagacttgtaaagaacaaatgtttcagttgaaacaaaaaaatggaggggatggtgggatcgaggctgttgaaataactgtttatgattattttgtcaatcaccgcaaaatcgatttacgatattctggtgatctgccatgcattaatgttgggaagccaaagcgccctacttatattcctcttgaggcgctttgttccctggtgtccctacaacgctataccaaagcactgtccacacttcaaaggtcttcactggtggagaaatcacggcagaagccgcaagaaaggatgactgttttatctagtgtaggtgctctgaagagcagcaagtatgatgctgaacctatgctacgctcgtgtggcatttcaatcaaccctagtttcacacaagtggaaggccgtgttctgcctgctccgaagctgaaagttggcaacggggaagatttcttcccaagaaatggacgttggaatttcaataacaagaaacttgtggagccatctaggatcgagaagtgggctgtcgtgaacttctcagctcgctgtgatatacgcaaccttgtacaaaatctcacaaaatgtgcagagatgaaaggaattcccatagaagatccttttgatgtatttgaggagaatccacaatcaagacgtgccccaccagtggttagagtggagaaaatgtttgagcaaattcagtctagacttcctgggcaaccaaagttcttactgtgcttgcttcctgagagaaagaattctgatatatatggcccatggaagcgcaaaaatcttgctgaatatggaattgtcactcagtgcattgcgcctcaaagagttaacgaccaatatattaccaatgttctcctgaagatcaatgcaaagcttggtgggttgaactctatgttggctgtggaacacgccccctcattacctcttgtgtcgaaggttcccacgcttatccttgggatggacgtgtcccatggctctcctgggcagtctgatgtcccttcaattgctgcggtagtcagctccaggcagtggcctttgatttctcgctatcgggcatgtgtgcgaacacaatccccaaagcttgagatgattgattcattatttaagcgagtgtctgagactgaggatgaaggaataattagggagcttctgttagacttttatgtgacttcaggaaaaaggaaacccgatcagatcatcatatttagagacggggtcagtgaatcacaattcaatcaggtcttgaatatcgaattggatcagataattgaggcgtgcaagtttcttgatgagaagtggtccccaacgtttgtggtaattgtagctcagaaaaaccaccacactaaatttttccaacctggatctcctgataatgtaccacctggtacgatcattgacaacaaagtctgccatccaagaaacaatgacttctatctctgtgctcatgctgggatgattgggactacaaggcctactcactaccatgttttgttagacgaggttggtttttcagcagatgatctgcaggaacttgtgcattccctctcatatgtataccaaagaagcacgactgccatctctgtagttgcaccaatctgctatgcccacctggcagctactcaaatgggccagtttatgaagtttgaagatacttctgagacttcctcaagccatggtggggtgacctctgcaggagctgttcctgtcccacagttgccgaggttgcaggagaaagtttgcaattccatgttcttttgttgagcatgctagcctttttactctctatgtaaataaaactagatagacagcaggtgtttggggtttctgaatttggtgcagccttgctgtggtggagtttagttgaacaccatgtgctggatccaagtcagcaaatatgtggtactacacctaggcatggtgatgccatttgacacgttttggaatcttggatgcagcctctaggtatggtcacttgctgatggcttgtcacagctttgcattttgttcagttcatgtttctgtacacaacgtcagcaagcaaaatccggtgcatgcaccatcctgggttatatggtattttgtaagtggatgattttgctttatgcatgcttgaaatttgtaatctgaatgctccaggaacgtcgttttctcaa

SEQ ID NO: 18 - DNA, 2790 bp, Lotus japonicus
atggacatggatccaaatgaacatgaaaatggaaatgggaatgggaatgggaatgaggatttgatgcctccgccaccaccaccccccattgtcccagctgatgtggaaccggtcaaggttgacctccttgacctaccccctgagccggtgaagaaaaaactacctacccggcttcccattgccaggaaaggtttgggttccaaagggaccaagctgccccttctcacaaatcactttaaagtaactgttgctaacagtgatggacatttcttccagtacagtgtggctctttcttacgaggatgggcgccctgtagaaggaaagggggttgggcgaaaagtgattgacaaggtgcaggagacatatggttctgagttgaatgggaaagactttgcctatgatggggagaagactttgtttaccattggttctcttgctcggaacaagcttgagtttacagttgttctagaggatgttatatctaacagaaacaatggaaactgcagccctgatggtgctagtacaaatgatagtgacaagaagagaatgagacggccttatcattccaaaactttcaaagttgagattagttttgctgcgaagattcccctgcaggcaattgtcaatgcccttcgtggtcaggaatctgagaattatcaggaagctataagagtgcttgatattatcctgagacaacatgctgctaagcaaggctgcttgcttgtgcgccagtcattcttccacaatgatccaaagaattatgcagatgttggaggtggtgtcctgggatgcagagggttccattccagctttagaaccacccaaagtggtctctccctgaacatagatgtctcaacaaccatgataattcaacctgggcctgtggtggacttcttaattgccaatcagaatgttagagatcctttttcacttgactgggcaaaggccaaaaggactctaaaaaatctgaggattaaagccagtccatccaatcaagagtacaaaatcacagggcttagcgaactaccatgcaaagagcagacttttactatgaagaaaaaaggtggaaacaatggggaggaggatgctactgaggaggagatcactgtttatgagtattttgttaattatcgaaagatagatctccggtactctgctgatcttccatgtataaatgtgggcaagccaaaacgccctacatatgtccctgttgagctttgctctttggtgtccttgcaacgttataccaaagctttaaccacacttcaaaggtcttcactagtagagaagtccaggcagaagcccctagagcggatgaatgtattgaatcaagctcttaaaaccagcaattatggcaatgaacccatgcttaaaaactgtggaattacaatagcttctggtttcactcaagttgagggtcgagtgctgcaagctccaaggttgaaatttggcaatggagaggatttcaacccgagaaatggaagatggaacttaaacaacaaaaaagtagtcaggccggcgaagatagaacactgggctgtggttaacttttctgctcgctgtgatgtacgagggcttgtgagggatttgatcaaatgtgcacgattgaaaggaattcctatagatgaaccttatgaagaaatatttgaagaaaatggtcagtttaggcgtgccccaccattggtcagagtggagaaaatgtttgagagaatccagaaggaacttcctggggcaccttcattccttctttgtcttcttcctgagcggaaaaactcggatctttatggtccatggaagaagaagaatctagctgagtatggaattgttactcagtgcatatctcctaccagggtcaacgatcaatatctcacaaatgttttgatgaagataaatgctaagctcggtgggttgaattcagtgttaggtgttgaaatgaatccttccattcctattgtttccaaagttcctaccatcattcttggtatggatgtgtctcatggctcgccagggcaatcagatattccttctattgctgcagttgtcagctccagagaatggcctctaatctcgaagtatagggcttgtgtccgtacccagtctcctaaggttgagatgattgataatttgttcaagcaagtgtctgaaaaggaagatgaaggaattataagggaacttctgattgatttttattcaagttctgggaagagaaaacccgataacataattatcttcagggatggggttagcgagtcccagttcaatcaagttttgaacattgaactaaatcaaatcattgaggcctgcaagtttctggatgaaacttggaaccccaaattcttggtgattgttgctcagaagaaccaccatacaaaattcttccagcctggttctcccgacaatgtgcctcctggaactgttattgataataaaatttgtcatcctcgaaataatgacttctatatgtgcgcacatgctggaatgattggtacaagcagacccactcactaccatgttctgcttgatgatattggtttttctcctgatgagctgcaagagctggtccattctctgtcttatgtttatcagaggagcaccactgccatttctgtggttgctccaatctgctatgctcatcttgctgcaacacagattgggcagtttatgaagttcgaggacaagtctgatacgtcttcgagccatggggggcttactgctgctggtgttgctcctgtcgtccctcagttgcctaagttgcaggatagcgtcagcagttctatgtttttctgttga

SEQ ID NO: 19 - protein, 16 aa, Artificial Sequence
  Synthetic peptide for AGO9 antibody generation
SSRNHAGNDTNDADRK

(19) United States
(12) Patent Application Publication
(10) Pub. No.: US 2013/0180001 A1
(43) Pub. Date: Jul. 11, 2013

(54) PLANTS THAT REPRODUCE VIA UNREDUCED GAMETES

(71) Applicants: Jean-Philippe Vielle-Calzada, Irapuato (MX); Vianey Olmedo-Monfil, Irapuato (MX)

(72) Inventors: Jean-Philippe Vielle-Calzada, Irapuato (MX); Vianey Olmedo-Monfil, Irapuato (MX)

(21) Appl. No.: 13/512,864

(22) PCT Filed: Nov. 30, 2010
(86) PCT No.: PCT/IB10/03216

§ 371 Date: Mar. 4, 2013

Related U.S. Application Data
(60) Provisional application No. 61/283,261, filed on Nov. 30, 2009.

(51) Int. Cl. C12N 15/82

(52) U.S. Cl. CPC C12N 15/82
USPC 800/260; 435/320.1; 800/298; 800/278

(57) ABSTRACT

System, including compositions and methods, for making and using plants that reproduce via unreduced gametes.

### CROSS-REFERENCE TO PRIORITY APPLICATION

This application is based upon and claims the benefit under all applicable national and international law, including 35 U.S.C. §119(e), of U.S. Provisional Patent Application Ser. No. 61/283,261, filed Nov. 30, 2009, which is incorporated herein by reference in its entirety for all purposes.

### INTRODUCTION

The majority of cultivated plants reproduce in a sexual manner. In sexual reproduction, the fusion of male and female gametes leads to the formation of seeds that combine maternal and paternal traits.

Although sexual reproduction predominates, plant species exist that reproduce themselves asexually through seeds. This asexual reproduction, termed apomixis, is a natural cloning process by which the female reproductive organ of a plant, the ovule, is able to form the embryonic portion of seeds, without the need for a genetic contribution from male gametes. In particular, an ovule of an apomictic plant produces one or more unreduced female gametes that form without undergoing meiosis. Accordingly, each unreduced female gamete maintains the somatic genotype of the parent plant when the gamete is incorporated into a seed and ultimately develops to form a child plant that is a clone of the parent.

The induction of apomixis in cultivated plants, such as in edible cereals, constitutes one of the most attractive challenges of agricultural biotechnology. Currently, the majority of improved, commercial seeds are the result of a long hybridization process in which certain plants that present desirable traits are selected and crossed to obtain seeds for an improved hybrid. However, the agronomic value of the improved hybrid is maintained only during one cultivation cycle. The natural sexuality of the hybrid causes the next generation to lose many of the desirable characteristics of the hybrid through separation of genetic traits. As a consequence, competitive producers find themselves obliged to buy seed year after year if they want to maintain high performances.

The ability to generate apomictic plant varieties would have tremendous commercial benefits. For example, creation of improved hybrids that exhibit a high rate of apomixis may, in some cases, make it possible for farmers to recurrently sow the seed produced by the improved hybrid, thereby maintaining the agronomic value of the seed for multiple generations (and potentially indefinitely). Also, by genetically fixing the agronomic value of any sexual cultivation, the ability to induce apomixis may encourage plant breeders to develop customized plant varieties adapted to specific environmental conditions. Additionally, the induction of apomixis offers the possibility of eliminating the use of costly cultivation techniques associated with vegetative reproduction of crop plants (e.g., potato, agave, and strawberry, among others). An ability to induce apomixis also may permit the preservation of individual plants with high rates of heterozygosis, such as vegetable species that are in danger of extinction.

Thus, there is a need for compositions and methods to force plants to execute one or more of the steps of apomixis, such as formation of unreduced female gametes by a parent plant. The formation of unreduced female gametes should avoid loss of desirable alleles during reproduction via seeds, because the somatic chromosomal constitution (and thus all alleles) of the parent plant would be transmitted to the next generation.

### SUMMARY

The present disclosure provides a system, including compositions and methods, for making and using plants that reproduce via unreduced gametes.

### BRIEF DESCRIPTION OF THE DRAWINGS

FIG. 1 is a flowchart illustrating reproduction of a diploid transgenic plant, which has been modified to reduce the activity of an endogenous, small RNA gene-silencing pathway, such that the plant forms unreduced, diploid female gametes and progeny that are diploid or polyploid, in accordance with aspects of the present disclosure.

FIG. 2 is a flowchart illustrating an exemplary method of transmitting an at least substantially complete, somatic set of chromosomes to a succeeding generation of a plant via seeds, in accordance with aspects of the present disclosure.

FIG. 3 is a schematic view of an exemplary nucleic acid construct for promoting formation of unreduced female gametes in plants, in accordance with aspects of the present disclosure.

### DETAILED DESCRIPTION

The present disclosure provides a system, including compositions and methods, for making and using plants that reproduce via unreduced gametes.

The present disclosure demonstrates that mutants disrupting small RNA gene-silencing, such as mutations in AGO4, AGO9, NRPD1, NRPD2, RDR2, RDR6, or SGS3, encourage formation of unreduced females gametes that have failed to undergo meiosis. The unreduced gametes may be formed via apospory, a component of asexual reproduction through seeds prevailing in many flowering species that produce unreduced female gametes from somatic cells in the ovule. Accordingly, epigenetic induction of at least one step of apomixis in a sexual plant may be achieved with a transgene that specifically reduces the activity of a small RNA gene-silencing pathway in reproductive tissue (e.g., ovules) of the plant. The process of small RNA gene-silencing may be used to attenuate itself by specifically inhibiting expression of at least one component of the gene silencing machinery.

A nucleic acid for plant transformation is provided. The nucleic acid may comprise a construct including a targeting sequence and a promoter sequence operatively coupled to the targeting sequence. The construct also may comprise any other suitable sequences, such as at least one selectable marker adapted to permit selective growth of a plant cell/plant and/or a bacterium carrying the nucleic acid.

The targeting sequence may encode an interfering RNA configured to specifically reduce expression of a component of a small RNA gene-silencing pathway in a plant. The component, such as AGO9, may (or may not) be naturally expressed specifically in reproductive tissue of a plant, for example, in the ovules, relative to at least most other plant tissues. The interfering RNA may be adapted specifically to reduce expression in a plant of an AGO4, AGO9, NRPD1, NRPD2, RDR2, RDR6, or SGS3 gene or polypeptide, or a combination of these genes or polypeptides, among others. The targeting sequence may include a sequence region, such as a sequence region of at least twenty consecutive nucleotides, that confers inhibition of expression of a plant AGO4, AGO9, NRPD1, NRPD2, RDR2, RDR6, or SGS3 gene or polypeptide (or a combination of these genes or polypeptides), and, optionally, that shows exact sequence identity to an expressed segment of the gene. The sequence region may have an antisense orientation with respect to the promoter sequence and may form an antisense part of an inverted repeat of the targeting sequence. The inverted repeat may form a hairpin structure when expressed as RNA. A loop of the hairpin structure may, for example, be an intron that is removed by splicing in a plant cell.

The construct may be configured to inhibit the small RNA gene-silencing pathway sufficiently to induce formation by the plant of one or more unreduced gametes. The pathway may be inhibited specifically by the construct in each ovule of the plant, relative to at least most other tissues of the plant. In some embodiments, the construct may be configured to express the interfering RNA specifically in each ovule of a plant relative to at least most other tissues of such plant. In some embodiments, the construct may confer expression of the interfering RNA that is conditional (e.g., with an activatable or repressible promoter sequence), such that a frequency of formation of unreduced gametes by the plant is adjustable.

A plant that reproduces through unreduced gametes and a method of producing the plant are provided. The plant may be transformed by the nucleic acid described above, such that the plant is a transgenic plant. Accordingly, the nucleic acid may be integrated into the plant's genome and/or may be stably heritable. The method may comprise selecting a founder plant; introducing the nucleic acid into the founder plant; and obtaining a transgenic descendant of the founder plant, with the transgenic descendant containing the nucleic acid and forming unreduced gametes.

A method of passing a somatic set of chromosomes to a succeeding generation of a plant is provided. A parent plant transformed with the nucleic acid may be selected. The parent plant may be cultivated such that the parent plant forms seeds. The seeds may be grown into one or more child plants each including an unreduced (somatic) set of chromosomes from the parent plant. In some cases, cultivation of the parent plant may include of mating the parent plant with a second plant to unite at least one male gamete of the second plant with an unreduced female gamete of the parent plant. In some cases, the second plant may be configured to provide at least substantially no chromosomal contribution to the child plants, such that the child plants are at least substantial clones of the parent plants. For example, the second plant may include a mutation in a CENH3 gene. In some embodiments, the child plants may have a higher ploidy than the parent plant. Reproduction through unreduced gametes should avoid loss of desirable alleles during reproduction via seeds, because the somatic set of chromosomes (and thus at least substantially all alleles) of the parent plant may be transmitted to the next generation.

Further aspects of the present disclosure are provided in the following sections: (I) abbreviations, (II) definitions, (III) system overview, (IV) exemplary nucleic acids for promoting formation of unreduced gametes, and (V) examples.

### I. ABBREVIATIONS

The various abbreviations used in the present disclosure generally are shorthand for terms recognized by those skilled in the art, consistent with the context in which each abbreviation is used. However, the following abbreviations may have additional and/or alternative meanings, as described below.

AGO4—ARGONAUTE 4

AGO9—ARGONAUTE 9

NRPD1—NUCLEAR RNA POLYMERASE D 1 (a DNA-dependent RNA Pol IV catalytic subunit)

NRPD2—NUCLEAR RNA POLYMERASE D 2 (a DNA-dependent RNA Pol IV catalytic subunit)

RDR2—RNA-DEPENDENT RNA POLYMERASE 2

RDR6—RNA-DEPENDENT RNA POLYMERASE 6

SGS3—SUPPRESSOR OF GENE SILENCING 3

An exemplary (Arabidopsis thaliana) mRNA sequence (as cDNA) and polypeptide sequence, respectively, for each of the above genes are presented in the associated Sequence Listing as SEQ ID NOS:1 and 2 (AGO4), SEQ ID NOS:3 and 4 (AGO9), SEQ ID NOS: 5 and 6 (NRPD1), SEQ ID NOS:7 and 8 (NRPD2), SEQ ID NOS:9 and 10 (RDR2), SEQ ID NOS:11 and 12 (RDR6), and SEQ ID NOS:13 and 14 (SGS3). Additional exemplary mRNA sequences (as cDNA) for AGO9 from other plant species are presented as SEQ ID NOS:45-18.

### II. DEFINITIONS

The various terms used in the present disclosure generally each have a meaning recognized by those skilled in the art, consistent with the context in which each term is used. However, the following terms may have additional and/or alternative meanings, as described below.

Unreduced gamete—a reproductive cell formed by a plant, having the same (“unreduced”) ploidy and/or genotype as somatic (sporophyte) cells of the plant, and capable of contributing genetic material for embryo formation. The gamete may be formed by and/or present in an ovule of the plant and may be described as a female gamete, whether or not the gamete is capable of uniting with a male gamete. A diploid plant produces unreduced gametes that are diploid, a triploid plant produces unreduced gametes that are triploid, as so on. An unreduced female gamete may unite with a male gamete to form a zygote that develops into an embryo, or, in some cases, may develop into an embryo without uniting with a male gamete. An unreduced female gamete may be described as having the same genotype as somatic cells of the plant, which means that at least substantially every allele of a somatic cell is also present in the gamete. In some examples, the chromosomal constitution of the gamete (or of a child plant or next generation) may be described as a somatic chromosomal constitution, which means that a copy of each and every somatic chromosome of the parent plant is present in the gamete (or child plant or next generation), with the linkage of alleles on each individual chromosome preserved when comparing somatic cells of the parent plant to the gamete (or child plant or next generation). A somatic chromosomal constitution may be generated in a gamete when no recombination occurs between homologous chromosomes during gamete formation.

Unreduced female gametes may be formed by diplospory or apospory, among others. The process of diplospory generates an unreduced gamete from a typical gamete precursor, a megaspore mother cell (MMC), which fails to undergo meiosis. The process of apospory generates an unreduced gamete by direct differentiation of a somatic cell into a gamete precursor, an MMC-like cell. The MMC-like cell generally is formed in a distinct site from the MMC (if present). Apospory may occur via a supernumerary gamete precursor while the usual gamete precursor undergoes meiosis (or apomeiosis).

Unreduced female gametes may be generated at any suitable frequency relative to total female gametes (unreduced and meiotically reduced). For example, the frequency of unreduced female gametes generated by an individual plant may be at least about 1%, 5%, 10%, or 25%, among others.

Apomixis—clonal reproduction through seeds. In apomixis, the embryo of a seed is formed with an unreduced maternal genome (from an unreduced female gamete) and with no paternal genome. Apomixis creates one or more seeds that germinate to produce one or more progeny which are at least nearly identical genetically to the mother plant. A plant that reproduces by apomixis forms viable apomictic seeds at a detectable frequency, with any suitable percentage of its seeds being apomictic, such as at least about 1%, 5%, 10%, 20%, 50%, or 100%, among others. An “apomictic seed” is a seed containing a viable embryo that is capable of developing into a plant that is at least nearly identical genetically to its progenitor (i.e., the parent plant). Plants that are at least nearly identical genetically to one another have respective genotypes that are indistinguishable from one another for at least about 95%, 99%, or 99.9% of the genes of the plants. Example 3 describes an example of apomixis in which a male gamete unites with an unreduced female gamete but makes no genetic contribution to the resulting embryo, which develops into a substantial clone of the mother plant.

RNA interference—a process of inhibiting gene expression in a targeted fashion using RNA mediators, which may be termed interfering RNAs. Interfering RNAs may include double-stranded RNAs, short interfering RNAs, micro RNAs, and/or the like. In some embodiments, the interfering RNA, as expressed or introduced, may be a double-stranded RNA, such as an RNA with a hairpin structure, which may be processed in the cell to form a small RNA (e.g., a short interfering RNA or a micro RNA). Small RNAs generally include RNAs of less than about 30 nucleotides, such as RNAs of 20, 21, 22, 23, 24, or 25 nucleotides, among others. RNA interference may inhibit gene expression before, during, and/or after transcription of a gene (i.e., by a transcriptional and/or a post-transcriptional mechanism), such as by gene modification (e.g., DNA/histone methylation), mRNA degradation, and/or inhibition of mRNA translation, among others.

RNA interference in plants is mediated by a small RNA gene-silencing pathway that inhibits expression of genes. The pathway, in plant ovules, relies on a number of genes/polypeptides to achieve gene silencing that encourages formation of reduced female gametes. These genes/polypeptides may be involved with formation of small RNAs and/or use of the small RNAs as guides to target particular genes and/or RNAs (such as for modification and/or degradation). These genes/polypeptides may include an ARGONAUTE family member (e.g., AGO4 or AGO9, among others), NRPD1, NRPD2, RDR2, RDR6, and/or SGS3, among others. Inhibition of the gene-silencing pathway in the ovule can result in formation of one or more unreduced female gametes. Exemplary polypeptides for AGO4, AGO9, NRPD1, NRPD2, RDR2, RDR6, and SGS3 from Arabidopsis thaliana are encoded by SEQ ID NOS:1, 3, 5, 7, 9, 11, and 13, respectively, and have amino acid sequences presented as SEQ ID NOS:2, 4, 6, 8, 10, 12, and 14, respectively. AGO4, AGO9, NRPD1, NRPD2, RDR2, RDR6, or SGS3 from other plant species may be identified as the polypeptide(s) in each species having the most similarity to SEQ ID NO:2, 4, 6, 8, 10, 12, or 14, respectively, or as a polypeptide having substantial similarity and/or identity to SEQ ID NO:2, 4, 6, 8, 10, 12, or 14, respectively.

An amount of identity or similarity between two polypeptides may be determined by the blastp algorithm (e.g., program BLASTP 2.2.18+), as described in the following two references, which are incorporated herein by reference: Stephen F. Altschul, et al. (1997), “Gapped BLAST and PSI-BLAST: a new generation of protein database search programs,” Constructs Res. 25:3389-3402; and Stephen, F. Altschul et al. (2005) “Protein database searches using compositionally adjusted substitution matrices,” FEBS J. 272:5101-5109. Examples of substantial similarity or identity include at least about 40%, 50%, 60%, 70%, or 80% sequence similarity or identity, a similarity score of at least about 200 or 250, and/or an E-Value of less than about 1e-40, 1e-60, or 1e-80, among others, using the blastp algorithm, with optimal alignment and, if needed, introduction of gaps.

Plant—a member of the Plantae kingdom of eukaryotic organisms, which may be described as a tree, bush, grass, shrub, herb, vine, moss, fern, algae, or a combination thereof, among others. A plant may (or may not) lack the capability for locomotive movement and generally possesses cell walls formed of cellulose. A plant may be capable of carrying out photosynthesis and may (or may not) be a vascular plant. In some embodiments, the plant may be an annual or a perennial. The plant may be a flowering plant (an angiosperm), such as a monocotyledon or a dicotyledon. In some embodiments, the plant may produce a grain, tuber, fruit, vegetable, nut, seed, fiber, oil, or a combination thereof, among others. Furthermore, the plant may be a crop plant. Exemplary crop plants that may be suitable for generation of transgenic plants according to the present disclosure include tobacco, potato, corn (maize), tomato, rice, wheat, alfalfa, soybean, and the like.

Transgenic plant—a plant comprising a nucleic acid construct. The construct may be integrated into the plants genome (i.e., nuclear or plastid genome), in some or at least substantially all of the cells of the plant. For example, the construct may be present in the plant's germline. Accordingly, the construct may be heritable, that is, inherited by at least one or more members, or at least substantially all members, of a succeeding generation of the plant. A transgenic plant that is “transformed” with a construct has been modified to contain the construct in the current generation or in any preceding generation(s) of the plant.

Nucleic acid—a compound comprising a chain of nucleotides. A nucleic acid may single-stranded or double stranded. A nucleic acid may have a natural or artificial (i.e., engineered) structure, or a combination thereof.

Gene—a nucleic acid or segment thereof that provides an expressible unit for expression of a polypeptide and/or a functional RNA (e.g., an interfering RNA). A gene thus may include a targeting region (also termed a targeting sequence) to define the sequence of the interfering RNA that is expressed and at least one transcriptional promoter (also termed a promoter sequence) operatively linked to the targeting region, to control (i.e., promote, drive, and/or regulate) transcription of the targeting region. A gene optionally may include one or more other control regions and/or untranslated regions, such as at least one 5′ leader sequence, intron, transcriptional terminator (also termed a terminator sequence), or any combination thereof, among others.

Construct—a nucleic acid created, at least in part, outside of plants using techniques of genetic engineering. A gene included in a construct may be termed a transgene.

Expression—a process by which a product, namely, an RNA and/or a polypeptide, is synthesized based on information encoded in a nucleic acid and/or gene, generally in the form of DNA (or RNA). Accordingly, the nucleic acid/gene may be expressed to form an RNA and/or polypeptide, which means that the RNA and/or polypeptide is expressed from the nucleic acid/gene.

### III. SYSTEM OVERVIEW

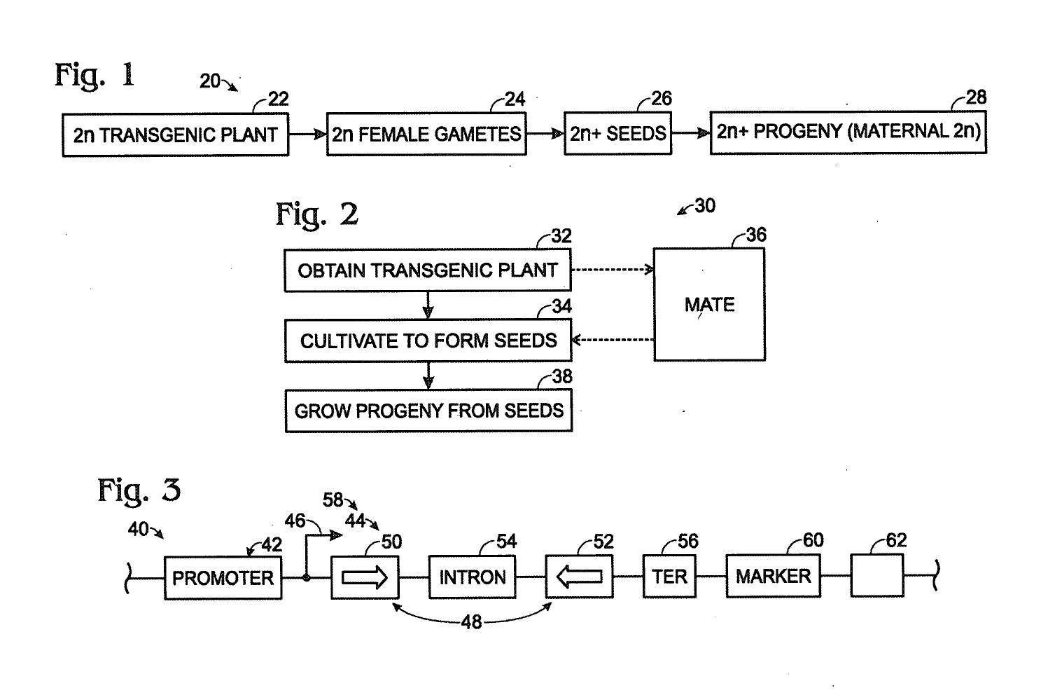

FIG. 1 shows a flowchart 20 illustrating reproduction of a diploid (2n) transgenic plant 22 constructed according to aspects of the present disclosure. In other examples, the transgenic plant may have any suitable ploidy, such as triploid (3n), tetraploid (4n), 5n, 6n, etc.

Plant 22 has been modified to reduce the activity of a small RNA gene-silencing pathway in ovules of the plant. For example, the plant may contain a construct (a transgene) that expresses an interfering RNA configured to specifically reduce expression of a component of a small RNA gene-silencing pathway that operates in ovules of the plant. The construct may be configured to inhibit operation of the pathway specifically in ovules relative to at least most other tissues of the plant. For example, the interfering RNA may be expressed from the construct in an ovule-specific pattern (i.e., expressed at a substantially higher level in ovules relative to at least most other tissues or the plant). Alternatively, or in the addition, the interfering RNA may be configured to inhibit expression of a gene/polypeptide (e.g., AGO9) that is normally expressed in an ovule-specific pattern relative to at least most of other tissues in the plant. Restricting the silencing action of the transgene to the ovule (and/or to reproductive tissue) may be important to avoid undesirable changes to the plant in nonreproductive plant tissues.

Due to inhibition of the gene-silencing pathway, the plant is encouraged to form unreduced female gametes 24. In other words, the ploidy of the transgenic plant is maintained in the unreduced gametes because there is no reduction of chromosome number through meiosis.

Seeds 26 are generated using female gametes 24. The seeds may have the same ploidy as gametes 24 and transgenic plant 22 (i.e., 2n in this case) or may have a higher ploidy (i.e., 3n, 4n, etc. in this case). The seeds may develop from gametes 24 without fertilization or as a result of fertilization with a male gamete of any suitable ploidy (e.g., 1n, 2n, 3n, 4n, etc.). The male gamete may be provided by the individual transgenic plant (self-fertilization) or another plant (cross-fertilization).

Progeny or child plants 28 may be produced by germinating seeds 26. The progeny may have the same ploidy as gametes 24 and transgenic plant 22, if there is no paternal contribution to the genotype of the progeny (e.g., see Example 3), or may have a higher ploidy with a paternal contribution. In any event, the progeny may contain at least substantially all of the alleles of parent transgenic plant 22 (in this case a 2n maternal contribution), since meiotic reduction did not occur during reproduction. Accordingly, the combination of alleles present in the parent plant may be transmitted to the next generation.

FIG. 2 shows a flowchart illustrating an exemplary method 30 of passing an at least substantially complete, somatic set of chromosomes to a succeeding generation of a plant via seeds. The steps presented here may be performed in any combination, in an order, and may be modified by or combined with any other aspect of the present disclosure.

A transgenic, parent plant may be obtained, indicated at 32. The transgenic plant may carry a transgene that expresses an interfering RNA configured to inhibit small RNA gene-silencing in ovules. The plant may be transformed with a nucleic acid containing the transgene and transformation may be performed in the current generation or any preceding generation of the plant.

The transgenic plant may be cultivated to form seeds, indicated at 34. The plant may form seeds by mating, indicated at 36, or parthogenetically, among others.

Progeny (child plants) may be grown from the seeds, indicated at 38. Each child plant may include an unreduced set of chromosomes from the parent plant.

### IV. EXEMPLARY NUCLEIC ACIDS FOR PROMOTING FORMATION OF UNREDUCED GAMETES

FIG. 3 shows a schematic view of a nucleic acid 40 for promoting apomixis in plants. Nucleic acid 40 may be constructed at least partially outside of plants. The nucleic acid may be DNA (or RNA), may be single- or double-stranded, may be linear or circular, or any combination thereof.

Nucleic acid 40 may include a gene that comprises a promoter sequence 42 operatively coupled to a targeting sequence 44. The gene may drive expression, indicated at 46, of the targeting sequence to produce an interfering RNA. The gene may be active in plants, that is, may be capable of causing a sufficient level of expression of the interfering RNA to achieve a phenotypic consequence, namely, formation of unreduced gametes. The promoter sequence may direct at least substantially ubiquitous expression or tissue-specific expression of the interfering RNA in a plant. Exemplary promoter sequences for widespread expression in a plant include promoters from Cauliflower Mosaic Virus (35S), rice actin, maize ubiquitin, etc. Tissue-specific promoters may direct expression selectively in reproductive tissue (e.g., ovules) of a plant relative to at least most other plant tissues. Exemplary tissue-specific promoters that may be suitable include pFM1 and pNuc1 ((1) PCT Patent Application Publication No. WO 2006/049482; (2) Huanca-Mamani W., Garcia-Aguilar M., León-Martinez G. Grossniklaus U, and Vielle-Calzada J-Ph. 2005. CHR11, a chromatin remodeling factor essential for nuclear proliferation during female gametogenesis in Arabidopsis, Proceedings of the National Academy of Sciences USA 102, 17231-17236; each of which is incorporated herein by reference). Another tissue-specific promoter that may be suitable, pFM2, is described in Example 1. A promoter sequence may provide conditional expression (i.e., inducible and/or repressible) or constitutive expression of the targeting sequence. Exemplary conditional promoters include chemically inducible and/or physically inducible promoters (e.g., inducible by a steroid hormone, auxin, tetracycline, metal, sugar starvation, ethanol, detergent, cis-jasmone, heat shock, etc.). The use of a conditional promoter may be advantageous to permit plant breeding (sexual reproduction), to generate a desired plant with a desired set of traits. Once the desired plant is generated, the conditional promoter may be induced (or derepressed), such that the set of traits is passed to a succeeding generation via unreduced gametes.

Targeting sequence 44 may include at least one targeting region 48 disposed in an antisense or a sense configuration with respect to promoter sequence 42. For example, targeting region 48 may include a pair of inverted repeats 50, 52 disposed in respective sense and antisense configurations and capable of forming a double-stranded RNA when expressed. The double-stranded RNA thus may form a stem of a stem-loop structure (a hairpin). In some embodiments, a loop 54 of the stem-loop structure may be formed by an intron.

In some examples, targeting sequence 44 may include a region from a plant AGO 4, AGO9, NRPD1, NRPD2, RDR2, RDR6, or SGS3 gene and/or mRNA (or cDNA). Exemplary mRNA/cDNA sequences for each of the above genes from Arabidopsis thaliana are presented as SEQ ID NOS:1, 3, 5, 7, 9, 11, and 13, respectively. The region may be of any suitable length, such as at least 20 consecutive nucleotides from the gene and/or mRNA thereof (e.g., a coding and/or untranslated region). The region may be from a plant ARGONAUTE 9 gene or mRNA, such as a coding and/or untranslated region from the gene/mRNA, or the like. Exemplary ARGONAUTE 9 sequences that may be suitable for designing a targeting sequence are provided by Arabidopsis thaliana (e.g., SEQ ID NO:3), Glycine max (soybean) (e.g., SEQ ID NO:15), Vitis vinifera (grape) (e.g., SEQ ID NO:16), Populus trichocarpa (poplar) (e.g., SEQ ID NO:17), or Lotus japonicus (a legume) (e.g., SEQ ID NO:18), among others.

Nucleic acid 40 also may incorporate a termination sequence 56 operatively coupled to the targeting sequence and positioned downstream thereof, with respect to promoter sequence 42. The termination sequence may encourage, and define a site of, transcriptional termination and/or post-transcriptional processing, such as polyadenylation, among others. Collectively, promoter sequence 42 and targeting sequence 44 (and, optionally, loop 54 and/or termination sequence 56) may form a chimeric gene or transgene 58 that expresses interfering RNA.

Nucleic acid 40 further may be equipped with any other suitable sequences, which may be outside of (or included in) chimeric gene 58. For example, the nucleic acid may include a selectable marker 60, which may permit selection for a growth advantage of plant cells and/or plants containing the nucleic acid, in the presence of a suitable selection agent/medium. The nucleic acid also or alternatively may comprise a selectable marker 62 for growth in bacteria (e.g., Agrobacterium tumefaciens) and/or a T-DNA sequence to promote plant transformation when exposed to Agrobacterium carrying the nucleic acid.

### V. EXAMPLES

The following examples describe selected aspects and embodiments of the present disclosure, such as exemplary compositions for, and methods of, making plants that reproduce via unreduced gametes. The examples are presented for illustration only and are not intended to define or limit the scope of the present disclosure.

### Example 1

### Promotion of Apospory by Gene Mutation

This example presents and describes exemplary data related to control of female gamete formation by a non-cell-autonomous small RNA pathway in Arabidopsis. The data demonstrates promotion of apospory by mutation of genes (e.g., AGO9, RDR6, and SGS3) involved in small RNA gene-silencing in plant reproductive tissue.

### A. SUMMARY

In the ovules of most sexual flowering plants female gametogenesis is initiated when a single gamete-precursor cell undergoes meiosis, giving rise to a single, functional, haploid product. Here we show that the Arabidopsis protein ARGONAUTE 9 (AGO9) controls female gametogenesis by restricting the specification of gamete precursors in a dosage-dependent, non-cell autonomous manner. Mutations in AGO9 lead to the differentiation of multiple female gamete precursors that are able to initiate gametogenesis. The AGO9 is not expressed in the gamete lineage; instead, it is expressed in cytoplasmic foci of somatic companion cells. Mutations in SUPPRESSOR OF GENE SILENCING 3 and RNA-DEPENDENT RNA POLYMERASE 6 exhibit an identical defect to ago9 mutants, indicating that the movement of small RNA silencing out of somatic companion cells is necessary for controlling the specification of gamete precursors. In the ovule, AGO9 preferentially interacts with 24 nt small RNAs (sRNAs) derived from transposable elements (TEs), and its activity is necessary to silence TEs in female gametes and their accessory cells. Our results show that AGO9-dependent sRNA silencing is crucial to specify cell fate in the Arabidopsis ovule, and that epigenetic reprogramming in companion cells is necessary for sRNA-dependent silencing in plant gametes.

### B. INTRODUCTION

The life cycle of flowering plants consists of a diploid (sporophytic) phase and two morphologically different haploid (gametophytic) phases occurring in specialized reproductive organs. By contrast to animals where meiotic products directly differentiate into functional reproductive cells, flowering plants require several mitotic divisions of the haploid precursors before differentiating their gametes. In the young ovule of Arabidopsis, a single sub-epidermal germ cell precursor (the megaspore mother cell, or MMC) differentiates and undergoes meiosis, giving rise to four haploid products (the megaspores). Only the proximal-most megaspore survives and gives rise to 8 nuclei after 3 mitotic divisions. Cellularization partitions the 8 nuclei into 7 cells: the egg and 2 synergid cells at the distal pole of the female gametophyte (or megagametophyte), a binucleated central cell, and 3 antipodal cells at the proximal pole. After fertilization of the egg and the central cell by 2 distinct sperm cells, the ovule develops into a seed. The establishment of the gametophytic phase presents an opportunity for natural selection to act on the haploid plant genome as an evolutionary driving force that could be at the origin of epigenetic mechanisms that ensure a tight regulation of plant reproductive development¹. Despite this early-acting selective pressure, there are numerous examples of developmental alternatives that suggest a flexible regulatory control of gamete formation. Because numerous sexual species and some mutants commonly exhibit more than one MMC²⁻⁴, and many others are able to form gametes without meiosis (by apomixis)⁵, it has been suggested that a group of somatic cells in the ovule is competent to respond to a local signal likely to play an important function in determination⁶; however, the genetic basis and molecular mechanisms controlling the specification of gamete precursors remain elusive.

### C. RESULTS

A large-scale transcriptional analysis by Massively Parallel Signature Sequencing (MPSS) showed that a gene encoding an ARGONAUTE (AGO) protein (At5g21150 or ARGONAUTE 9) is highly expressed in ovules and anthers of Arabidopsis but absent from other vegetative or reproductive organs. ATH1 microarray expression profiles and reverse transcriptase PCR (RT-PCR) confirmed that ARGONAUTE 9 (AGO9) is only expressed in ovules and anthers before and after fertilization. To determine the reproductive pattern of AGO9 expression, we performed in situ hybridization in developing ovules and anthers of wild-type plants using both whole-mount gynoecia as well as completely sectioned inflorescences. AGO9 mRNA was absent from vegetative tissues (leaves, stems, roots) or developing sepals or petals. Before differentiation of the MMC, AGO9 mRNA was abundantly localized in the nascent ovule primordium, including cells of the epidermal layer (L1), the sub-epidermal layer (L2) and the most inner cell layers (L3), and weakly in the septum. At meiosis, AGO9 mRNA became restricted to a cluster of L1 and L2 cells located at the distal (micropylar) region of the developing ovule, but was absent from the MMC or the megaspores. During female gametogenesis, AGO9 mRNA was abundantly localized in the distal and proximal pole of the ovule, but not within the developing female gametophyte. These results indicate that prior to fertilization AGO9 is expressed in female sporophytic companion cells of the developing ovule, but not in the female gametes.

In both plants and animals, AGO proteins are known to cleave endogenous mRNAs during either microRNA (miRNA) or short interfering RNA (siRNA)-guided post-transcriptional silencing⁷. They bind to short interfering RNAs and microRNAs through a conserved PAZ domain, and, in animals, they assemble into a multi-subunit RNA-induced silencing complex (RISC) responsible for degrading a target mRNA or repressing its translation^{8,9}. To elucidate the function of AGO9 in Arabidopsis, individuals from 3 independent insertional lines harboring T-DNA elements within the coding region of the AGO9 gene were phenotypically analyzed at all stages of ovule development¹⁰. Whereas 94.2% of pre-meiotic ovules showed a single MMC in wild-type plants, and 5.8% exhibited 2 MMCs; however, only one of the latter underwent gametogeness since twin developing female gametophytes were never observed. All ago9 insertional lines were fertile and did not show signs of ovule or seed abortion; however, in contrast to wild-type plants, the pre-meiotic ovule primordia of heterozygous ago9/+ individuals—including allele ago9-2 that was previously reported as having no defective phenotype¹¹—showed several abnormally enlarged sub-epidermal cells reminiscent of the MMC. In ago9/+ individuals, the ovules exhibited up to 6 cells containing a conspicuous nucleus and nucleolus at a frequency of 30.29%, indicating that ago9 alleles are dominant and affect early cell differentiation in the developing ovule. In homozygous ago9/ago9 individuals, the percentage of ovule primordia showing more than one enlarged cell was of 37.16% to 47.7%, depending on the allele that was tested (Table 1).

TABLE 1
Genetic analysis of insertional ago9 mutants in Arabidopsis.

| | | | More than 1 |
|---|---|---|---|
| Allele | Genotype | Single MMC | MMC-like cell |
| ago9-3 | ago9-3/ago9-3 | 208 | 123 (37.16%) |
| | ago9-3/+ | 214 | 93 (30.29%) |
| | ago9-3^{m}/+^{p}/+^{p} | 286 | 47 (14.11%) |
| | +^{m}/+^{m}/ago9-3^{p} | 241 | 74 (23.49%) |
| ago9-4 | ago9-4/ago9-4 | 139 | 118 (45.9%) |
| ago9-2 | ago9-2/ago9-2 | 162 | 148 (47.7%) |
| wild-type | +/+ | 292 | 18 (5.8%) |

To determine if dosage effects could be responsible for the mutant phenotype in heterozygous plants, the presence of abnormally enlarged cells was scored in F1 individuals resulting from reciprocal crosses between diploid heterozygous ago9-3/+ and tetraploid (4n) wild-type plants (Table 1). Triploid (3n) individuals that had 2 wild-type and one mutant ago9-3 allele showed 14.11% to 23.49% of abnormal ovules, a value intermediate between diploid plants carrying a single ago9-3 allele and wild-type. These results suggest that a dosage-dependent mechanism is responsible for the mutant ago9-3/+ phenotype.

In Arabidopsis, female meiosis occurs before cytokinesis, and subsequent cellularization gives rise to a tetrad of haploid cells¹². While 3 of the meiotically derived cells degenerate, the most proximal one enlarges and ultimately forms a single female gametophyte in each ovule. No molecular marker exclusively expressed in the MMC has been reported, but the pattern of callose deposition is a reliable method to determine cell identity at pre-meiotic stages. To determine whether one or several of the enlarged cells present in ago9-3 ovules are capable of undergoing meiosis, we analyzed callose deposition in more than 100 wild-type or homozygous ago9-3 cleared ovules at pre-meiotic, meiotic or post-meiotic stages, and conducted detailed comparisons of sub-epidermal cell morphology. In agreement with previous descriptions, wild-type ovules showed patches of callose in the MMC prior to the initiation of meiosis. After meiosis, callose was deposited in transverse walls between the functional megaspore and its degenerated sister cells. In pre-meiotic ago9-3 ovules, abnormally enlarged cells often showed patches of callose deposits reminiscent of those found in the wild-type MMC. During meiosis, callose was only detected in the intermediate walls of a single cell and the degenerated neighboring cells, but not in the closely associated abnormally enlarged cells. This pattern persisted following meiosis. These results show that several enlarged cells in ago9-3 ovules acquire a germ cell precursor identity, but that a single one undergoes meiosis and gives rise to a functional haploid megaspore, indicating that the activity of AGO9 is necessary to restrict differentiation to a single sub-epidermal cell in the pre-meiotic ovule.

Following meiosis, ago9-3 ovules showed persistent gamete precursors adjacent to meiotic products, including the 3 degenerated megaspores and the functional megaspore. To determine the identity and assess the developmental potential of extranumerary gamete precursor cells in mutant ovules, we examined the expression of the pFM2 marker, which is initially expressed post-meiotically in the functional megaspore and subsequently in the developing female gametophyte. In contrast to pFM1 that occasionally drives weak reporter gene expression in somatic cells surrounding the functional megaspore¹³, pFM2 is an ideal marker to characterize cells that have acquired a functional identity after meiosis because its activity is strictly restricted to the functional megaspore but it is not expressed in the MMC or in the 3 meiotically-derived degenerated megaspores. At subsequent developmental stages, pFM2 is only active in the developing female gametophyte. In ago9-3 ovules, pFM2 expression was initially observed following meiosis in the functional megaspore but also in a cluster of adjacent cells that forms the nucellus and includes the abnormal gamete precursors. In all ago9-3 ovules observed, more than 4 cells showed strong GUS expression at post-meiotic stages, indicating that at least some of the cells that express pFM2 have a somatic origin. In agreement with callose deposition, pFM2 expression was absent at pre-meiotic stages, indicating that defective ago9-3 individuals differentiate additional MMC-like cells that persist in the developing ovule adjacent to the meiotic products and subsequently acquire a functional megaspore identity without undergoing meiosis. At subsequent stages of development and in agreement with the presence of extranumerary pre-meiotic precursors (Table 1), ago9-3 individuals exhibited a quite unusual phenotype of 2 independent female gametophytes developing in the same ovule at a frequency of 44.03% (n=243). Crosses of ago9-3 plants with individuals expressing the pFM1 or pFM2 marker revealed that both acquire a female gametophyte identity. These results suggest that abnormal somatic precursor cells are able to initiate gametogenesis without undergoing meiosis.

To determine the pattern of AGO9 localization, we generated a peptide antibody against a specific epitope of 16 amino acids located in positions 139 to 154 of the N-terminal protein region. In Western blots, an AGO9 protein of the expected 100.5 kDa size was detected in wild-type developing gynoecia but not in 1-week old seedlings, developing rosette leaves or siliques 7 days after pollination. By examining the subcellular localization of AGO9 at early stages of ovule formation, we determined that AGO9 was initially expressed in somatic cells of the epidermal (L1) layer located in the apical region of the pre-meiotic ovule primordium, but not in the MMC. Interestingly, we observed AGO9 in cytoplasmic foci reminiscent of P-bodies or stress granules present in the cytoplasm of animal cells. While this pattern of activity persisted throughout meiosis, a few L2 cells expressed AGO9 after megaspore degeneration, at the onset of female gametogenesis; however, AGO9 did not localize in the haploid megaspores or the developing female gametophyte before of after cellularization. In ovules containing a female gametophyte at the 4-nuclear stage, AGO9 was localized in the outer integumentary cells, but also in the periphery of the endothelium, at the sporophyte-gametophyte cellular boundary. L1 and L2 cells of the mature outer integument often show polarized AGO9 localization associated to transverse cell walls. In anthers, AGO9 was also localized in the cytoplasm of microsporocytes following meiosis, and later in the cytoplasm of the vegetative cell but not in the sperm cells. Ovules or pollen of ago9-3 individuals did not show AGO9 expression, confirming that the antibody exclusively recognized AGO9 and not a different protein of the AGO family. Overall, these results indicate that AGO9 is preferentially expressed in reproductive companion cells but not in the associated male or female gametes or their precursors.

The specific epidermal (L1) pattern of protein localization at pre-meiotic stages combined with the presence of sub-epidermal germ cell precursors in mutant individuals suggests that AGO9 acts in a non-cell-autonomous manner to repress germ cell precursor commitment in the ovule. In Arabidopsis, only trans-acting small interfering RNAs (ta-siRNAs) are known to move as signal molecules and cause gene silencing beyond their cellular sites of initiation^{14,15}, resembling both viral and transgene siRNAs in this respect¹⁶. In each case biogenesis depends on transcription by RNA-DEPENDENT RNA POLYMERASE 6 (RDR6) that converts their single-stranded RNA precursors into double-stranded RNA in a pathway that is also dependent on the function of the putative RNA binding protein SUPRESSOR OF GENE SILENCING 3 (SGS3)^{17,18}. The extent of gene silencing movement outside their site of initiation also depends upon the activity of RDR6¹⁹. To determine if the non-cell-autonomous function of AGO9 could be associated with a ta-siRNA pathway, we characterized ovule development and female gametogenesis in homozygous sgs3-11 and rdr6-11 individuals. Although both sgs3 and rdr6 mutants show seedling and floral defects characterized by leaf curling and limited stamen elongation¹⁷, their possible role during gamete formation has not been investigated. Both sgs3-11 and rdr6-11 plants showed an identical phenotype to ago9 mutants with additional germ cell precursors differentiating in the pre-meiotic ovule. In rdr6-11 plants, post-meiotic ovules showed 2 independently developing female gametophytes at a frequency of 43.3% (n=224). Crosses of rdr6-11 plants to individuals expressing the pFM2 marker indicate that abnormal somatic precursor cells are also able to form female gametes. These results support the hypothesis that AGO9, SGS3, and RDR6 control germ cell precursor commitment by acting in a non-cell-autonomous small RNA-dependent pathway in the developing ovule of Arabidopsis.

To identify the nature of AGO9-interacting small RNAs, and due to extremely low small RNA yields obtained in pilot experiments conducted with hundreds of female reproductive organs, 12,000 wild-type developing gynoecia containing ovules up to the 4-nucleate stage of gametogenesis were isolated and used for total protein extraction. To identify and classify the small RNA fraction associated with the complex, previously eluted and gel-purified small RNAs were ligated with adaptors at their 5′ and 3′ ends, converted to cDNA products, and subsequently cloned and sequenced by Sanger methods. Whereas immunopurifications conducted with the pre-immune serum did not yield any bacterial clones containing endogenous Arabidopsis sequences, we obtained a total 2,552 sequences representing 344 distinct small RNAs from immunopurifications conducted with the AGO9 antibody. After removal of the adaptors, 2508 small RNA sequences (98% of total) could be mapped to the Arabidopsis nuclear genome and categorized based on their location and function. Although the majority are 24 nt in length (79.1%), 8.9% are 21 to 22 nt long, indicating that in the ovule AGO9 can also interact with 21 nt bona fide small RNAs, including microRNAs (miRNAs). The majority of 24 nt sequences derive from transposable elements (TEs) belonging to distinct families of retrotransposons: Gypsy (23%) Athila (9.3%), CACTA (5.5%), and less frequently LINE or Mutator. Whereas all sequences mapping to Gypsy TEs belong to the AtGP1 sub-family, 3% represent mapped siRNA sequences shown to be dependent on RNA Polymerase IV (PolIV) for their biogenesis²⁰. An additional 17.4% maps to genomic signatures assigned to other families containing nested components of Gypsy, Athila or CACTA TEs. In contrast, 21 nt small RNAs preferentially derive from previously characterized miRNAs (3.2%)—including miR167 that is known to act in the ovule²¹—and protein-coding genes (14.5%). These results show that the primary target of AGO9-dependent silencing in the ovule of Arabidopsis are TEs.

Previous studies have shown that some TEs that are active in mature pollen grains are not expressed in developing or fully differentiated ovules of Arabidopsis²². To determine if AGO9 is necessary for the inactivation of these TEs in the ovule, we crossed lines containing enhancer traps (ET) that have tagged a specific TE to homozygous ago9 individuals. In agreement with previous results, no GUS expression was observed in the ovule or female gametophyte of ET lines present in a wild-type genetic background. By contrast, heterozygous ago9/+ individuals containing an ET within either an Athila, LINE, or Atlantys retrotransposon showed strong GUS staining in the egg and synergid cells of the mature female gametophyte prior to pollination. These results not only confirm that AGO9 is necessary for TE inactivation in the ovule, but also show that one of its targets is the egg and synergid cells (the egg apparatus) before fertilization.

The discovery that a small RNA-dependent mechanism controls female gametophyte development in Arabidopsis indicates that AGO9 interacts with an SGS3/RDR6-dependent pathway and is crucial to specify female germ cell precursors in the ovule. Our results suggest that AGO9 acts in a dosage-dependent, non-cell-autonomous manner to repress the reproductive commitment of sub-epidermal somatic cells by inactivation of target transcripts, either transcriptionally or posttranscriptionally²³. By preferentially interacting with small RNAs derived from TEs and silencing their activity in the female gametophyte, the function of AGO9 is reminiscent of the PIWI subclass of ARGONAUTE proteins that are necessary to maintain transposon silencing in the germline genome of invertebrates and mammals; in most animals PIWI defective individuals are also affected in germ cell differentiation²⁴. AGO9 acts in neighboring cells and not directly in pre-meiotic or meiotic products, highly reminiscent of short interfering RNA (siRNA) biogenesis in pollen grains and confirming previous results showing that epigenetic reprogramming in companion cells is a conserved mechanism for small RNA silencing of TEs in both male and female gametes²². Some maternal siRNA sequences found in the endosperm²⁰ and 24 nt siRNA found in pollen²² resemble AGO9-interacting sRNAs, raising the possibility that AGO9 may also contribute to these populations in a non-autonomous way.

Our results also indicate that mutants in this AGO9-dependent ta-siRNA pathway allow somatically derived gamete precursors to undergo female gametogenesis. This mechanism is reminiscent of apospory, a component of asexual reproduction through seeds (apomixis) prevailing in many flowering species that produce unreduced female gametes from somatic cells in the ovule⁵. Our findings open new venues to investigate the genetic basis and molecular mechanisms that control cell fate in the ovule, offering new possibilities to explore the epigenetic induction of apomixis in sexual plants.

### D. MATERIALS AND METHODS

Material and growth conditions. For all experiments we used Arabidopsis thaliana of ecotype Columbia 0 (Col-0). Insertional mutant lines were ago9-2 (SALK_{—}112059), ago9-3 (SAIL_{—}34_G10), agog-4 (SAIL_{—}260_A03) (ago9-1 was also analyzed but not quantified and showed the same phenotype). Insertion sites were verified, and homozygous lines selected by RT-PCR. For detailed description of mutant stocks, enhancer trap lines, transgenic lines and DNA constructs, see Methods. Seeds were sterilized with 100% ethanol and germinated under the stable long day (16 h light/8 h dark) conditions at 22° C. Seedlings were planted and grown under controlled greenhouse conditions (24° C.).

Histological analysis. Cleared ovules and histochemical GUS analysis was performed as described²⁵. Callose analysis was performed as described²⁶ with minor modifications described in Methods.

Immunoblot and immunoprecipitation. A peptide, SSRNHAGNDTNDADRK (SEQ ID NO:19), was used to generate a specific AGO9 antibody (Invitrogen, Carlsbad Calif.). Immunopurification of AGO9-small RNAs complex was performed as described²⁷.

Cloning and genomic analysis of small RNAs. After sequencing, small RNA reads were filtered and sequences were mapped to the Arabidopsis genome (http://www.arabidopsis.org).

### E. REFERENCES

1. Evans, M. M & Walbot, V. Unique features of the plant life cycle and their consequences. Nat. Rev. Genet. 4, 369-379 (2003).
2. Maheswari, P. An introduction to the embryology of the angiosperms. Published by McGraw-Hill Book Company, New York (1950).
3. Sheridan, W F., Avalkina N A, Shamrov I I, Batygina T B, & Golubovskaya I N. The mac1 gene: controlling the commitment to the meiotic pathway in maize. Genetics 153, 933-941 (1999).
4. Nonomura, K., Miyoshi K., Eiguchi M., Suzuki T., Miyao A., Hirochika H., & Kurata N. The MSP1 gene is necessary to restrict the number of cells entering into male and female sporogenesis and to initiate anther wall formation in rice. Plant Cell 15, 1728-1739 (2003).
5. Bicknell, R. A. & Koltunow, A. M. Understanding apomixis: recent advances and remaining conundrums. Plant Cell 16 Suppl, S228-S245 (2004).
6. Grossniklaus, U. & Schneitz, K. The molecular and genetic basis of ovule and megagametophyte development. Sem. Cell. Dev. Biol. 9, 227-238 (1998).
7. Baumberger, N & Baulcombe, D. C. Arabidopsis ARGONAUTE1 is an RNA Slicer that selectively recruits microRNAs and short interfering RNAs. PNAS 102, 11928-11933 (2005)
8. Schwarz, D. S., Hutvágner G., Du T., Xu Z., Aronin N. & Zamore P D. Asymmetry in the assembly of the RNAi enzyme complex. Cell 115, 199-208 (2003).
9. Pham J. W., Pellino J. L., Lee Y. S. & Carthew R. W., Sontheimer E. J.

A Dicer-2-dependent 80s complex cleaves targeted mRNAs during RNAi in Drosophila. Cell 117, 83-94 (2004).
10. Alonso, J. M. et al. Genome-wide insertional mutagenesis of Arabidopsis thaliana. Science 301, 653-657 (2003).
11. Takeda A., Iwasaki S., Watanabe T., Utsumi M. & Watanabe Y. The mechanism selecting the guide strand from small RNA duplexes is different among argonaute proteins. Plant Cell Physiol. 49, 493-500 (2008).
12. Webb, M. C. & Gunning, B. E. S. Embryo sac development in Arabidopsis thaliana: Megasporogenesis, including the microtubular cytoskeletoin. Sex. Plant Reprod. 3, 244-258 (1990).
13. Huanca-Mamani W., Garcia-Aguilar M., León-Martinez G., Grossniklaus U., & Vielle-Calzada J P. CHR11, a chromatin-remodeling factor essential for nuclear proliferation during female gametogenesis in Arabidopsis thaliana. PNAS 102, 17231-17236 (2005).
14. Chitwood D. H., Nogueira F. T., Howell M. D., Montgomery T. A., Carrington J. C., & Timmermans M. G. Pattern formation via small RNA mobility. Genes Dev. 23, 549-554 (2009).
15: Schwab R., Maizel A., Ruiz-Ferrer V., Garcia D., Bayer M., Crespi M., Voinnet O., & Martienssen R. A. Endogenous tasiRNAs mediate non-cell autonomous effects on gene regulation in Arabidopsis thaliana. PLoS One 19, e5980 (2009).
16. Voinnet, O. Non-cell autonomous RNA silencing., FEBS Lett. 579, 5858-5871 (2005).
17. Peragine A., Yoshikawa M., Wu G., Albrecht H. L., & Poethig R. S. SGS3 and SGS2/SDE1/RDR6 are required for juvenile development and the production of trans-acting siRNAs in Arabidopsis. Genes Dev. 18, 2368-2379 (2004).
18. Yoshikawa, M., Peragine, A., Park M. Y., & Poethig R. S. A pathway for the biogenesis of trans-acting siRNAs in Arabidopsis. Genes Dev. 15, 2164-2175 (2005).
19. Himber C., Dunoyer P., Moissiard G., Ritzenthaler C., Voinnet O. Transitivity-dependent and -independent cell-to-cell movement of RNA silencing. EMBO J. 22, 4523-4533 (2003).
20. Mosher R. A., Melnyk C. W., Kelly K. A., Dunn R. M., Studholme D. J., Baulcombe D. C. Uniparental expression of PolIV-dependent siRNAs in developing endosperm of Arabidopsis. Nature 460, 283-286 (2009).
21. Wu M. F., Tian Q., & Reed J. W. Arabidopsis microRNA167 controls patterns of ARF6 and ARF8 expression, and regulates both female and male reproduction. Development 133, 4211-4218 (2006).
22. Slotkin R. K., Vaughn M., Borges F., Tanurdzić M., Becker J. D., Feijó J. A., & Martienssen R. A. Epigenetic reprogramming and small RNA silencing of transposable elements in pollen. Cell 136, 461-472 (2009).
23. Matzke M, Kanno T, Huettel B, Daxinger L, Matzke A J. Targets of RNA-directed DNA methylation. Curr Op. Plant Biol. 10, 512-519 (2007).
24. C. Klattenhoff, C. & Theurkauf W. Biogenesis and germline functions of piRNAs. Development 135, 3-9 (2008).
25. Vielle-Calzada, J. P., Baskar, R., & Grossniklaus, U. Delayed activation of the paternal genome during seed development. Nature 404, 91-94 (2000).
26. Siddiqi, I., Ganesh, G., Grossniklaus, U. and Subbiah, V. The dyad gene is required for progresión through female meiosis in Arabidopsis. Development 127, 197-207 (2000).
27. Qi Y., Denli A. M., & Hannon G. J. Biochemical specialization within Arabidopsis RNA silencing pathways. Mol Cell. 19, 421-428 (2005).

Further aspects of promoting apospory are described in U.S. Provisional Patent Application Ser. No. 61/283,261, filed Nov. 30, 2009, which is incorporated herein by reference.

### Example 2

### Promotion of Apospory by Inhibiting AGO9 Expression

This example describes exemplary data related to promotion of apospory by inhibiting AGO9 expression through RNA interference.

### A. RESULTS

Apospory can be induced in sexual crops by inhibiting ARGONAUTE 9 (AGO9) expression. The importance of AGO9 function in promoting sexual reproduction can be demonstrated using an RNA interference approach. To produce an AGO9 interference construct, a 275-bp fragment of an AGO9 cDNA was cloned into a pFGC5941 RNAi vector in both sense and antisense orientations. (The pFGC5941 RNAi vector is described in Kerschen et al., 2004, and is a publically available RNAi vector developed by the group of Rich Jorgensen at the University of Arizona.)

The AGO9 interference construct, pFGC5941, that was used to conduct these experiments contains a 35S promoter of Cauliflower mosaic virus (CaMV35S) and was modified such that the 35S promoter drives transcription of a partial AGO9 sequence cloned in both sense and antisense orientations and separated by an intron of the chalcone synthase gene. After formation of a hairpin RNA structure, the resulting double-stranded RNA transcripts may cause posttranscriptional silencing of endogenous gene activity (Waterhouse et al., 1998; Chuang and Meyerowitz, 2000; Smith et al., 2000). A detailed analysis of CaMV35S promoter activity during ovule development has shown that this promoter is active in sporophytic (somatic diploid) cells of Arabidopsis but not in the gamete (haploid) lineage. We reasoned that AGO9 transcripts localized in sporophytic cells can be the target of RNAi-dependent silencing driven by CaMV35S.

Wild-type Arabidopsis thaliana plants of the ecotype Columbia were transformed with the AGO9 interference construct. After floral-dipping transformation, 45 primary transformants were generated, none of which showed visible defects during vegetative growth, root development, or floral organogenesis. However, all adult T1 transformants showed defects identical to those of insertional ago9 plants (see Example 1), but at significantly higher frequencies. The percentage of ovules showing extranumerary germ precursor cells was of 70 to 92% in 10 RNAi-AGO9 T1 plants tested. To determine a possible relationship between a decrease in AGO9 transcript levels and the defective phenotype, RNA was extracted from developing used for RT-PCR experiments. All 10 plants tested showed absence of AGO9 expression during ovule development, indicating that the interfering RNA produced from the AGO9 interference construct silenced AGO9 expression in the ovule.

The same experiments can be performed in members of the Solanaceae, including tobacco and tomato.

### B. REFERENCES

Chuang, C. F., and Meyerowitz, E. (2000). Specific and heritable genetic interference by double-stranded RNA in Arabidopsis thaliana. Proc. Natl. Acad. Sci. USA 97, 4985-4990.
Kerschen, A., Napoli, C. A., Jorgensen, R. A., and Müller, A. E. (2004). Effectiveness of RNA interference in transgenic plants. FEBS Lett. 566, 223-228.
Smith, N. A., Singh, S. P., Wang, M. B., Stoutjesdijk, P. A., Green, A. G., and Waterhouse, P. M. (2000). Total silencing by intron-spliced hairpin RNAs. Nature 407, 319-320.
Waterhouse, P. M., Graham, M. W., and Wang, M. B. (1998). Virus resistance and gene silencing in plants can be induced by simultaneous expression of sense and antisense RNA. Proc. Natl. Acad. Sci. USA 95, 13959-13964.

### Example 3

### Synthetic Clonal Reproduction Through Seeds Using Argonaute9

This example describes exemplary data related to clonal reproduction through seeds using an ago9 mutant mated with a tailswap mutant.

### A. ABSTRACT

Cloning through seeds has potential revolutionary applications in agriculture because its introduction into sexual crops would allow perpetuation of any elite heterozygous genotype. Asexual reproduction through seeds, or apomixis, results in progeny that are genetic clones of the maternal parent. However, despite the occurrence of apomixis in hundreds of plant species, very few crop species reproduce via apomixis and attempts to introduce this trait by crossing have failed. An alternative approach is to de novo engineer the production of clonal seeds¹. We previously showed that one major element of apomixis, the formation of functional unreduced gametes that are genetically identical to the parent plant (apomeiosis) could be induced in the sexual plant Arabidopsis thaliana argonaute9 mutants². However, these gametes participate in the normal sexual process of fertilization, leading to an increase in ploidy at the next generation. Here we demonstrate the conversion of apomeiotic gametes into clonal seeds by crossing to a strain whose chromosomes are engineered to be eliminated after fertilization. We crossed argonaute9² plants to a cenh3-1 mutant expressing altered CENH3 proteins³. A subset of F1 progeny were clones of their parent, mimicking progeny resulting from natural apomixis. These results demonstrate that clonal reproduction through seeds can be achieved in sexual plants.

### B. INTRODUCTION

The life cycle of flowering plants consists of a diploid (sporophytic) phase and two morphologically different haploid (gametophytic) phases occurring in specialized reproductive organs. By contrast to animals where meiotic products directly differentiate into functional reproductive cells, flowering plants require several mitotic divisions of the haploid precursors before differentiating their gametes. In the young ovule of Arabidopsis, a single sub-epidermal germ cell precursor (the megaspore mother cell, or MMC) differentiates and undergoes meiosis, giving rise to four haploid products (the megaspores). Only the proximal-most megaspore survives and gives rise to 8 nuclei after 3 mitotic divisions. Cellularization partitions the 8 nuclei into 7 cells: the egg and 2 synergid cells at the distal pole of the female gametophyte (or megagametophyte), a binucleated central cell, and 3 antipodal cells at the proximal pole. After fertilization of the egg and the central cell by 2 distinct sperm cells, the ovule develops into a seed. The establishment of the gametophytic phase presents an opportunity for natural selection to act on the haploid plant genome as an evolutionary driving force that could be at the origin of epigenetic mechanisms that ensure a tight regulation of plant reproductive development¹. Despite this early-acting selective pressure, there are numerous examples of developmental alternatives that suggest a flexible regulatory control of gamete formation. Because numerous sexual species commonly exhibit more than one MMC^{2,3}, and many others are able to form gametes without meiosis (by diplosporous or aposporous apomixis)⁴, it has been suggested that a group of somatic cells in the ovule is competent to respond to a local signal likely to play an important function in determination⁵; however, the genetic basis and molecular mechanisms controlling the specification of gamete precursors remain elusive. Apomixis is hypothesized to have originated as a modified form of sexual reproduction that has undergone deregulation of some key steps, either by mutagenesis or epigenetic changes. Functional models for apomixis genes suggest they are alleles of genes involved in sexual reproduction that are ectopically or heterochronically active as a result of mutation or epigenetic modifications relative to the sexual allele. There is currently no direct evidence to support these models in apomictic or sexual species.

### C. THE ARGONAUTE 9 PATHWAY AND ITS BEARING ON APOMIXIS

We have shown that the Arabidopsis “slicer” protein ARGONAUTE 9 (AGO9) controls female gametogenesis by restricting the specification of gamete precursors in a dosage-dependent, non-cell autonomous manner (e.g., see Examples 1 and 2). Mutations in AGO9 lead to the differentiation of multiple female gamete precursors that are each able to initiate gametogenesis. The AGO9 is not expressed in the gamete lineage; instead, it is expressed in somatic companion cells. Strikingly, mutations in SUPPRESSOR OF GENE SILENCING 3 and RNA-DEPENDENT RNA POLYMERASE 6 exhibit an identical defect to ago9 mutants, suggesting that the movement of small RNA silencing out of somatic companion cells is necessary for controlling the specification of gamete precursors. Although in the ovule AGO9 preferentially interacts with 24 nt small RNAs (sRNAs) derived from transposable elements (TEs), and its activity is necessary to silence TEs in female gametes and their accessory cells, it is not yet clear if RNA-dependent silencing of repetitive elements is directly related to the ago9 phenotype, or if this phenotype is dependent on other small RNAs that also interact with AGO9, such as microRNAs or other 21 nt siRNAs. Our results show that AGO9-dependent sRNA silencing is crucial to specify cell fate in the Arabidopsis ovule, and that epigenetic reprogramming in companion cells is necessary for sRNA-dependent silencing in plant gametes. We have also demonstrated the presence of additional gametic precursors with rdr6, rdr2, sgs3, nprd1, and nprd2 mutants.

We have extended our results by showing that most 24 nt sRNA interactors of AGO9 map to the pericentromeric regions of all 5 Arabidopsis chromosomes, indicating a possible link between AGO9 function and heterochromatin formation and perhaps meiosis (Durán-Figueroa and Vielle-Calzada, Plant Signaling and Behavior Vol. 5 Issue 1 November 2010).

We also showed that mutations in ago9 result in a significant frequency of viable unreduced female gametes that maintain the maternal chromosomal constitution; this frequency ranges from 8 to 17% depending on allelic variants and environmental conditions. The differentiation of viable unreduced female gametophytes in these mutants is reminiscent of apospory in apomictic species. In apospory, the initiation of sexual reproduction is concomitant with the differentiation of somatic aposporous initial cells in the vicinity of a sexually derived cell (the functional megaspore). As in apospory, abnormal unreduced gametic precursors in ago9 undergo 2 nuclear mitosis and subsequent expansion before differentiating a 4-cellular female gametophyte containing 2 synergids, the egg cell, and single polar nucleus. The degree to which the sexual and abnormal “aposporous-like” pathways interact remains unclear.

Additionally, a second mutation in a different ARGONAUTE gene (ARGONAUTE 4 or AGO4) shows functional redundancy with AGO9. Double homozygous ago9 ago4 individuals show a dramatic exacerbation of gametic precursors proliferating in the developing ovule, with abnormal unreduced megaspore differentiation in epidermal as well as funicular cells of the ovule primordium. Sometimes they also show aberrant meiotic configurations in which functional megaspore specification among haploid derived nuclei appears to be misregulated.

As an alternative to seed development without fertilization, we reasoned that apomeiotic gametes could be turned into seeds by fertilizing them with a strain whose chromosomes are eliminated from the resultant progeny. Directional genome elimination occurs in certain wide crosses (both interspecific and intergeneric), and leads to the formation of haploid plants. It has been shown that altering the centromeric-specific histone variant CENH3 by the so-called “tailswap” line in Arabidopsis leads to preferential elimination of chromosomes from the cenh3 mutant parent following a cross to wild type³. As the ago9 plants were from a mixed No-0 and Col-0 background, and tailswap was pure Col-0 we could trace the origin of the chromosomes in the F1 progeny. The ago9×tailswap crosses gave an average of 14 seeds per fruit, 21.1% being fully maternal diploids lacking the paternal contribution. With 292 total plants analyzed, the crosses gave a germination rate of 92%, a triploid rate of 16.6%, and a clone rate of 21.1%. Furthermore, these diploid eliminants systematically kept the heterozygosity of the mother plant for all tested loci. For all crosses these results rule out post elimination doubling following fertilization of a haploid gamete and show that genome elimination took place after fertilization of an unreduced female gamete, and that resulting plants were clones of the maternal parent. Taken together, these results demonstrate engineering of clonal propagation through seed in a manner akin to the outcome of diplosporous or aposporous apomixis. Our results provide a proof of principle demonstration for synthesis of apomixis in a sexual plant.

### D. MATERIALS AND METHODS

Plant material and growth conditions. Plants were grown in artificial soil mix at 20° C. under fluorescent lighting. Wild type and mutant strains of Arabidopsis were obtained from ABRC, Ohio or NASC, UK. ago9-3 is an insertional mutant (SAIL_{—}34_G10). ago9-3 were crossed to the No-0 ecotype to generate populations that were heterozygous for markers across the genome.

Ploidy analysis. Isolation of nuclei for fluorescence activated cell sorting (FACS) was performed as described⁴. FACS analysis was carried out using an internal diploid and tetraploid control to unambiguously identify diploid plants. MiMe and osd1 offspring ploidy analyses were performed by flow cytometry and chromosome spreads.

### E. REFERENCES

1. Spillane, C., Curtis, M. D. & Grossniklaus, U. Apomixis technology development-virgin births in farmers' fields? Nat Biotechnol 22, 687-91 (2004).
2. Olmedo-Monfil, V. et al. Control of female gamete formation by a small RNA pathway in Arabidopsis. Nature 464, 628-32 (2010).
3. Ravi, M. & Chan, S. W. Haploid plants produced by centromere-mediated genome elimination. Nature 464, 615-8 (2010).
4. d'Erfurth, I. et al. Turning meiosis into mitosis. PLoS Biol 7, e1000124 (2009).

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A nucleic acid for plant transformation, comprising:
a construct including (a) a targeting sequence encoding an interfering RNA configured to specifically reduce expression of a component of a small RNA gene-silencing pathway in a plant and (b) a promoter sequence operatively coupled to the targeting sequence and active or activatable in an ovule of the plant,
wherein the construct is configured to inhibit the small RNA gene-silencing pathway specifically in each ovule of the plant, relative to at least most other tissues of the plant, and to inhibit such pathway sufficiently to induce formation by the plant of one or more unreduced gametes.

2. The nucleic acid of claim 1, wherein the construct is configured to express the interfering RNA specifically in each ovule of a plant relative to at least most other tissues of such plant.

3. The nucleic acid of claim 2, wherein the component of a small RNA gene-silencing pathway is expressed with a substantially nonspecific distribution in the plant.

4. The nucleic acid of claim 1, wherein the component of a small RNA gene-silencing pathway is expressed specifically in each ovule of the plant relative to at least most other tissues of the plant, if the plant does not contain the construct.

5. The nucleic acid of claim 4, wherein the ARGONAUTE polypeptide is AGO4 or AGO9.

6. The nucleic acid of claim 5, wherein the ARGONAUTE polypeptide is AGO9.

7. The nucleic acid of claim 6, wherein the construct is configured to express the interfering RNA with a substantially nonspecific distribution in the plant.

8. The nucleic acid of claim 1, wherein the interfering RNA is configured to specifically reduce an expressed level of an AGO4, AGO9, NRPD1, NRPD2, RDR2, RDR6, or SGS3 polypeptide, or a combination thereof, relative to at least most other polypeptides in the plant.

9. The nucleic acid of claim 8, wherein the targeting sequence includes an inverted repeat of a nucleotide sequence from AGO4, AGO9, NRPD1, NRPD2, RDR2, RDR6, or SGS3, or a combination thereof.

10. The nucleic acid of claim 9, wherein the nucleotide sequence is at least about 20 nucleotides in length and is provided by AGO9.

11. The nucleic acid of claim 10, wherein the nucleotide sequence is included in at least one of SEQ ID NOS:3 and 15-18.

12. The nucleic acid of claim 1, wherein expression of the interfering RNA is conditional, such that a frequency of formation of unreduced gametes by the plant is adjustable.

13. A plant comprising the nucleic acid of claim 1 and having a capability conferred by the nucleic acid to form unreduced gametes.

14. A method of creating a transgenic plant, comprising:
selecting a founder plant;
introducing the nucleic acid of claim 1 into the founder plant; and
obtaining a transgenic descendant of the founder plant, the transgenic descendant containing the nucleic acid of claim 1 and being capable of forming unreduced gametes.

15. The method of claim 14, wherein the step of selecting a founder plant includes a step of selecting a founder plant from a variety having one or more desirable traits, and wherein the step of obtaining a transgenic descendant of the founder plant includes a step of obtaining a transgenic descendant having the one or more desirable traits.

16. A method of passing a somatic set of chromosomes to a succeeding generation of a plant, comprising:
selecting a parent plant transformed with the nucleic acid of claim 1;
cultivating the parent plant such that the parent plant forms seeds; and
growing the seeds into one or more child plants each including an unreduced set of chromosomes from the parent plant.

17. The method of claim 16, wherein the step of cultivating includes a step of mating the parent plant with a second plant to unite at least one male gamete of the second plant with an unreduced female gamete of the parent plant, and wherein the step of mating is configured to provide at least substantially no chromosomal contribution from the second plant to the child plants, such that the child plants are at least substantial clones of the parent plant.

18. The method of claim 17, wherein the second plant includes a mutation of a CENH3 gene.

19. The method of claim 16, wherein the child plants have a higher ploidy than the parent plant.

20. A nucleic acid for plant transformation, comprising:
a construct including (a) a targeting sequence encoding an interfering RNA configured to specifically reduce expression of at least one protein selected from the group consisting of AGO4, AGO9, NRPD1, NRPD2, RDR2, RDR6, and SGS3 in a plant and (b) a promoter sequence operatively coupled to the targeting sequence and active or activatable in an ovule of the plant,
wherein the construct is configured to inhibit expression of the at least one polypeptide specifically relative to other polypeptides of the plant, and to inhibit such expression sufficiently to induce formation, in an ovule of the plant, of one or more unreduced gametes each including a somatic ploidy of the plant.

21. The nucleic acid of claim 20, wherein the construct is configured to inhibit expression of the at least one polypeptide specifically in each ovule of the plant relative to at least most other tissues of the plant.

22. The nucleic acid of claim 20, wherein the construct is configured to promote formation of unreduced gametes by apospory.

23. The nucleic of claim 20, wherein the promoter sequence confers ovule-specific expression of the interfering RNA.

24. The nucleic acid of claim 20, wherein the interfering RNA is configured to specifically reduce expression of AGO9.
